(19) United States
(12) Patent Application Publication
(10) Pub. No.: US 2023/0128137 A1
(43) Pub. Date: Apr. 27, 2023

(54) BENZO FIVE-MEMBERED CYCLIC COMPOUND

(71) Applicant: MEDSHINE DISCOVERY INC., Nanjing, Jiangsu (CN)

(72) Inventors: Hongjian WANG, Shanghai (CN); Wenyuan QIAN, Shanghai (CN); Ming ZHANG, Shanghai (CN); Jian LI, Shanghai (CN); Shuhui CHEN, Shanghai (CN)

(21) Appl. No.: 17/910,536

(22) PCT Filed: Mar. 8, 2021
(86) PCT No.: PCT/CN2021/079579

§ 371 Date: Sep. 9, 2022

(30) Foreign Application Priority Data
Mar. 12, 2020 (CN) 202010171071.3

(51) Int. Cl. C07D 471/04; A61P 35/00

(52) U.S. Cl. CPC C07D 471/04; A61P 35/00

(57) ABSTRACT

A benzo five-membered cyclic compound represented by formula (I) or a pharmaceutically acceptable salt thereof has a significant effect in inhibiting the activity of anti-apoptotic Bcl-2 protein.

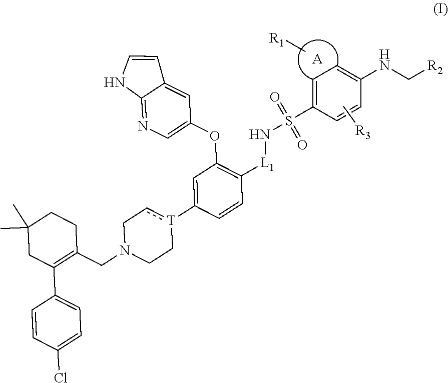

The present invention claims the right of the following priority for: CN202010171071.3, Mar. 12, 2020.

### TECHNICAL FIELD

The present disclosure relates to a benzo five-membered cyclic compound, and relates to a compound represented by formula (I) or a pharmaceutically acceptable salt thereof.

### BACKGROUND

Bcl-2 protein family is the central regulatory factor of apoptosis and programmed cell death, and the cell death may occur in response to internal pressure signals or environmental signals. In the life cycle of any organism, proliferation must be balanced with apoptosis to ensure proper development and proper mature physiological cells and organ functions. In highly proliferative tissues such as bone marrow, the balance between proliferation and apoptosis is particularly important. The change of apoptosis pathway may lead to cancer, and resistance to apoptosis has been considered as a sign of human cancer nearly 20 years ago. Members of the Bcl-2 protein family can inhibit or activate apoptosis. Bcl-2 family proteins can be divided into three categories: proteins that inhibit apoptosis, including Bcl-2, Bcl-xL and Mcl-1, etc.; proteins that promote apoptosis, including Bak, Bax, etc.; and other pro-apoptotic proteins containing BH3 domain only such as Bad, Puma, etc. The balance between Bcl-2 and Bak proteins at the checkpoint of cell death signals determines the survival or apoptosis of cells.

Bcl-2 is able to prevent the release of cytochrome c from mitochondria into the cytoplasm, thereby inhibiting apoptosis; and Bcl-2 is also able to inhibit the changes in mitochondrial permeability and affect the formation of macropores, thereby inhibiting apoptosis. In normal body tissues, the distribution of Bcl-2 is relatively limited, mainly in early embryonic tissues, mature lymphocytes, proliferative active epithelial cells and neurons, etc. The expression of Bcl-2 is enhanced in many tumors such as breast cancer, neuroblastoma, nasopharyngeal cancer, prostate cancer, bladder cancer, lung cancer, gastric cancer and colon cancer, etc. Overexpression of Bcl-2, one of the most common changes in malignant lymphoid tumors, disrupts the balance between pre-apoptotic and anti-apoptotic proteins. Bcl-2 gene is a proto-oncogene that can inhibit cell death caused by a variety of factors, including inhibition of target cell apoptosis caused by most chemotherapeutic drugs, making tumors drug resistant. Therefore, Bcl-2 protein inhibitors can selectively exert anti-tumor effects, and the inhibition of Bcl-2 activity can be used for the treatment of hematological malignancies and various solid tumors.

### CONTENT OF THE PRESENT INVENTION

The present disclosure provides a compound represented by formula (I) or a pharmaceutically acceptable salt thereof,

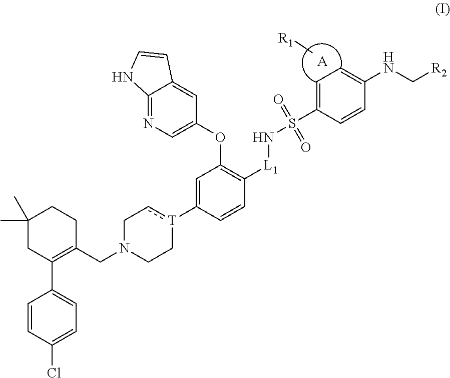

wherein,

when T is N, [symbol] is selected from a single bond;

when T is C, [symbol] is selected from a double bond;

ring A is selected from

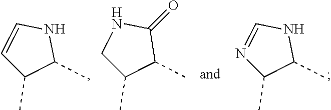

R₁ is selected from H and C₁₋₃ alkyl, and the C₁₋₃ alkyl is optionally substituted by one Rₐ;

R₂ is selected from oxacyclohexyl;

R₃ is selected from H, F, Cl, Br, I, NO₂ and CN;

L₁ is selected from a single bond and —C(═O)—;

Rₐ is selected from H and

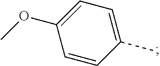

In some embodiments of the present disclosure, the R₁ is selected from H and CH₃, and the CH₃ is optionally substituted by one R^{a}, and the other variables are as defined herein.

In some embodiments of the present disclosure, the R₁ is selected from H, CH₃ and

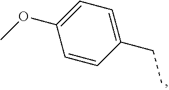

and the other variables are as defined herein.

In some embodiments of the present disclosure, the R₂ is selected from

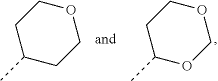

and the other variables are as defined herein.

In some embodiments of the present disclosure, the R₃ is selected from H and NO₂, and the other variables are as defined herein.

In some embodiments of the present disclosure, the compound is selected from

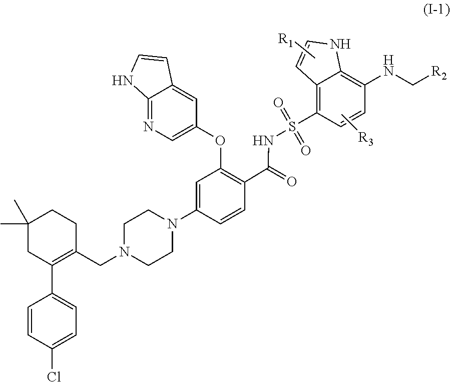

wherein, R₁, R₂ and R₃ are as defined herein.

In some embodiments of the present disclosure, the compound is selected from

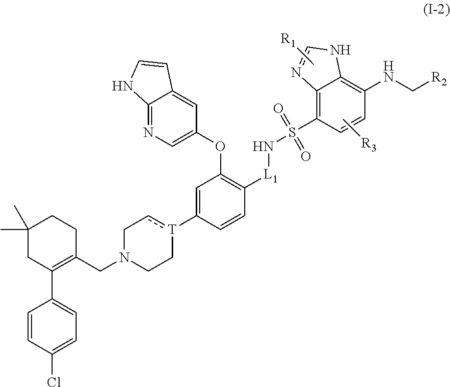

wherein,

when T is N, [symbol] is selected from a single bond;

when T is C, [symbol] is selected from a double bond;

R₁, R₂, R₃ and L₁ are as defined herein.

In some embodiments of the present disclosure, the compound is selected from

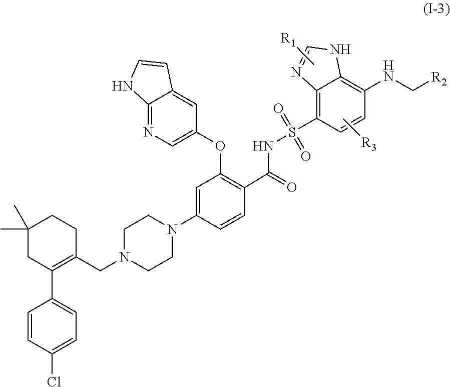

wherein, R₁, R₂ and R₃ are as defined herein.

In some embodiments of the present disclosure, the compound is selected from

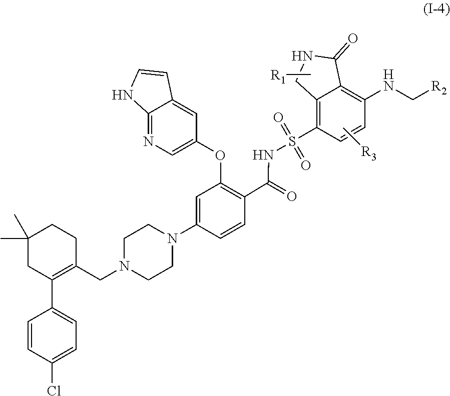

wherein, R₁, R₂ and R₃ are as defined herein.

In some embodiments of the present disclosure, the structural moiety

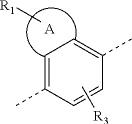

is selected from

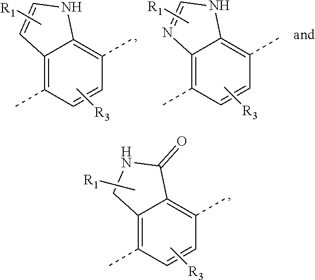

and the other variables are as defined herein.

In some embodiments of the present disclosure, the structural moiety

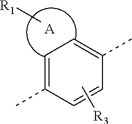

is selected from

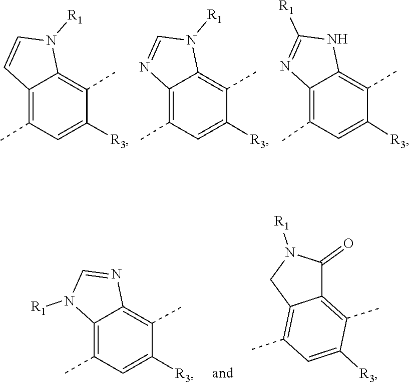

and the other variables are as defined herein.

In some embodiments of the present disclosure, the structural moiety

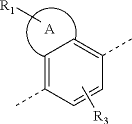

is selected from

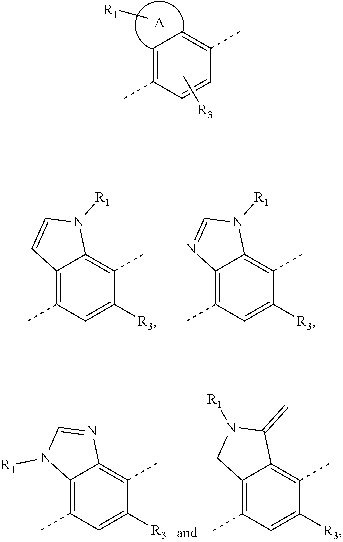

and the other variables are as defined herein.

In some embodiments of the present disclosure, the structural moiety

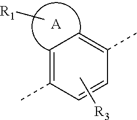

is selected from

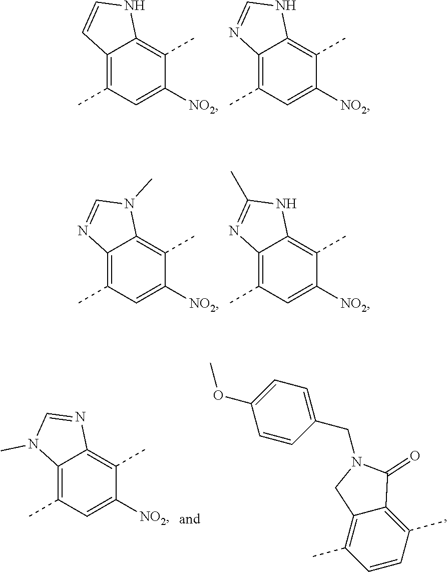

and the other variables are as defined herein.

The present disclosure also has some embodiments derived from any combination of the above variables.

The present disclosure also provides a compound represented by the following formula or a pharmaceutically acceptable salt thereof,

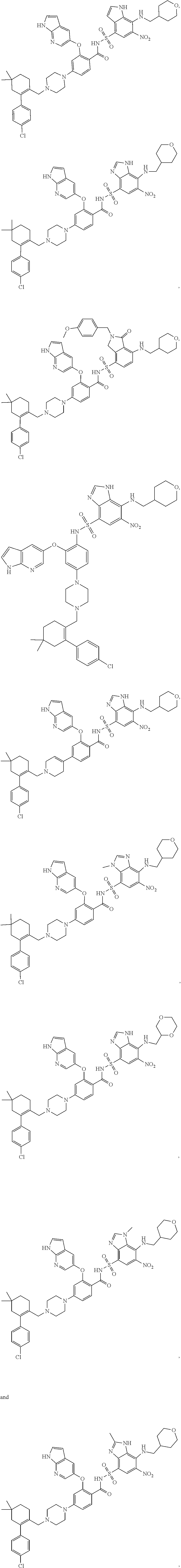

In some embodiments of the present disclosure, provided is a use of the compound or the pharmaceutically acceptable salt thereof in the manufacture of a medicament related to Bcl-2 inhibitors.

In some embodiments of the present disclosure, the medicament related to Bcl-2 inhibitors is a medicament for the treatment of hematological malignancies and solid tumors.

Technical Effects

Compared with anti-apoptotic Bcl-2 protein and anti-apoptotic Bcl-xL protein, the compound of the present disclosure exhibits good selectivity, and has a significant effect in inhibiting the activity of anti-apoptotic Bcl-2 protein; has a good metabolic stability of liver microsomes in humans, SD rats, CD-1 mice and beagle dogs and the species difference is small; has a good pharmacokinetic properties in CD-1 mice in vivo, supporting the oral administration route; has a significant inhibitory effect on the division and proliferation of RS4;11 cells, and can significantly inhibit tumor growth. Related medicaments can be used to treat a variety of diseases, such as malignant hemangioma, solid tumors, autoimmune diseases, cardiovascular diseases, and neurodegenerative diseases, especially have great application prospects in the treatment of tumor diseases.

### Definition and Description

Unless otherwise specified, the following terms and phrases when used herein have the following meanings. A specific term or phrase should not be considered indefinite or unclear in the absence of a particular definition, but should be understood in the ordinary sense. When a trade name appears herein, it is intended to refer to its corresponding commodity or active ingredient thereof.

The term “pharmaceutically acceptable” is used herein in terms of those compounds, materials, compositions, and/or dosage forms, which are suitable for use in contact with human and animal tissues within the scope of reliable medical judgment, with no excessive toxicity, irritation, an allergic reaction or other problems or complications, commensurate with a reasonable benefit/risk ratio.

The term “pharmaceutically acceptable salt” refers to a salt of the compound of the present disclosure that is prepared by reacting the compound having a specific substituent of the present disclosure with a relatively non-toxic acid or base. When the compound of the present disclosure contains a relatively acidic functional group, a base addition salt can be obtained by bringing the compound into contact with a sufficient amount of base in a pure solution or a suitable inert solvent. The pharmaceutically acceptable base addition salt includes a salt of sodium, potassium, calcium, ammonium, organic amine or magnesium, or similar salts. When the compound of the present disclosure contains a relatively basic functional group, an acid addition salt can be obtained by bringing the compound into contact with a sufficient amount of acid in a pure solution or a suitable inert solvent. Examples of the pharmaceutically acceptable acid addition salt include an inorganic acid salt, wherein the inorganic acid includes, for example, hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphorous acid, and the like; and an organic acid salt, wherein the organic acid includes, for example, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, and methanesulfonic acid, and the like; and salts of amino acid (such as arginine and the like), and a salt of an organic acid such as glucuronic acid and the like. Certain specific compounds of the present disclosure contain both basic and acidic functional groups, thus can be converted to any base or acid addition salt.

The pharmaceutically acceptable salt of the present disclosure can be prepared from the parent compound that contains an acidic or basic moiety by conventional chemical method. Generally, such salt can be prepared by reacting the free acid or base form of the compound with a stoichiometric amount of an appropriate base or acid in water or an organic solvent or a mixture thereof.

The compounds of the present disclosure may exist in specific geometric or stereoisomeric forms. The present disclosure contemplates all such compounds, including cis and trans isomers, (−)- and (+)-enantiomers, (R)- and (S)-enantiomers, diastereomers isomers, (D)-isomers, (L)-isomers, and racemic and other mixtures thereof, such as enantiomers or diastereomeric enriched mixtures, all of which are within the scope of the present disclosure. Additional asymmetric carbon atoms may be present in substituents such as alkyl. All these isomers and their mixtures are included within the scope of the present disclosure.

Unless otherwise specified, the term “enantiomer” or “optical isomer” refers to stereoisomers that are mirror images of each other.

Unless otherwise specified, the term “cis-trans isomer” or “geometric isomer” is caused by the inability to rotate freely of double bonds or single bonds of ring-forming carbon atoms.

Unless otherwise specified, the term “diastereomer” refers to a stereoisomer in which a molecule has two or more chiral centers and the relationship between the molecules is not mirror images.

Unless otherwise specified, “(+)” refers to dextrorotation, “(−)” refers to levorotation, and or “(±)” refers to racemic.

Unless otherwise specified, the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) and a straight dashed bond ( [symbol] ), a wave line ( [symbol] ) is used to represent a wedged solid bond ( [symbol] ) or a wedged dashed bond ( [symbol] ), or the wave line ( [symbol] ) is used to represent a straight solid bond ( [symbol] ) or a straight dashed bond ( [symbol] ).

Unless otherwise specified, the terms “enriched in one isomer”, “enriched in isomers”, “enriched in one enantiomer” or “enriched in enantiomers” refer to the content of one of the isomers or enantiomers is less than 100%, and the content of the isomer or enantiomer is greater than or equal to 60%, or greater than or equal to 70%, or greater than or equal to 80%, or greater than or equal to 90%, or greater than or equal to 95%, or greater than or equal to 96%, or greater than or equal to 97%, or greater than or equal to 98%, or greater than or equal to 99%, or greater than or equal to 99.5%, or greater than or equal to 99.6%, or greater than or equal to 99.7%, or greater than or equal to 99.8%, or greater than or equal to 99.9%.

Unless otherwise specified, the term “isomer excess” or “enantiomeric excess” refers to the differential value between the relative percentages of two isomers or two enantiomers. For example, if the content of one isomer or enantiomer is 90%, and the content of the other isomer or enantiomer is 10%, the isomer or enantiomer excess (ee value) is 80%.

Optically active (R)- and (S)-isomer, or D and L isomer can be prepared using chiral synthesis or chiral reagents or other conventional techniques. If one kind of enantiomer of certain compound of the present disclosure is to be obtained, the pure desired enantiomer can be obtained by asymmetric synthesis or derivative action of chiral auxiliary followed by separating the resulting diastereomeric mixture and cleaving the auxiliary group. Alternatively, when the molecule contains a basic functional group (such as amino) or an acidic functional group (such as carboxyl), the compound reacts with an appropriate optically active acid or base to form a salt of the diastereomeric isomer which is then subjected to diastereomeric resolution through the conventional method in the art to give the pure enantiomer. In addition, the enantiomer and the diastereoisomer are generally isolated through chromatography which uses a chiral stationary phase and optionally combines with a chemical derivative method (such as carbamate generated from amine). The compound of the present disclosure may contain an unnatural proportion of atomic isotope at one or more than one atom(s) that constitute the compound. For example, the compound can be radiolabeled with a radioactive isotope, such as tritium (³H), iodine-125 (¹²⁵I) or C-14 (¹⁴C). For another example, deuterated drugs can be formed by replacing hydrogen with heavy hydrogen, the bond formed by deuterium and carbon is stronger than that of ordinary hydrogen and carbon, compared with non-deuterated drugs, deuterated drugs have the advantages of reduced toxic and side effects, increased drug stability, enhanced efficacy, extended biological half-life of drugs, etc. All isotopic variations of the compound of the present disclosure, whether radioactive or not, are encompassed within the scope of the present disclosure.

The term “substituted” means one or more than one hydrogen atom(s) on a specific atom are substituted with the substituent, including deuterium and hydrogen variables, as long as the valence of the specific atom is normal and the substituted compound is stable. When the substituent is an oxygen (i.e., ═O), it means two hydrogen atoms are substituted. Positions on an aromatic ring cannot be substituted with a ketone. The term “optionally substituted” means an atom can be substituted with a substituent or not, unless otherwise specified, the type and number of the substituent may be arbitrary as long as being chemically achievable.

When any variable (such as R) occurs in the constitution or structure of the compound more than once, the definition of the variable at each occurrence is independent. Thus, for example, if a group is substituted with 0-2 R, the group can be optionally substituted with up to two R, wherein the definition of R at each occurrence is independent. Moreover, a combination of the substituent and/or the variant thereof is allowed only when the combination results in a stable compound.

When the number of a linking group is 0, such as —(CRR)₀—, it means that the linking group is a single bond.

When the number of a substituent is 0, it means that the substituent does not exist, for example, -A-(R)₀ means that the structure is actually A.

When a substituent is vacant, it means that the substituent does not exist, for example, when X is vacant in A-X, the structure of A-X is actually A.

When one of the variables is selected from a single bond, it means that the two groups linked by the single bond are connected directly. For example, when L in A-L-Z represents a single bond, the structure of A-L-Z is actually A-Z.

When a bond of a substituent can be cross-linked to two or more atoms on a ring, such a substituent can be bonded to any atom on the ring, for example, a structural unit

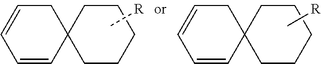

means that a substituent R can be substituted at any position on cyclohexyl or cyclohexadiene. When the enumerative substituent does not indicate by which atom it is linked to the group to be substituted, such substituent can be bonded by any atom thereof. For example, when pyridyl acts as a substituent, it can be linked to the group to be substituted by any carbon atom on the pyridine ring.

When the enumerative linking group does not indicate the direction for linking, the direction for linking is arbitrary, for example, the linking group L contained in

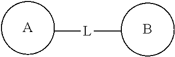

is -M-W—, then -M-W— can link ring A and ring B to form

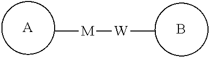

in the direction same as left-to-right reading order, and form

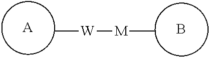

in the direction contrary to left-to-right reading order. A combination of the linking groups, substituents and/or variables thereof is allowed only when such combination can result in a stable compound.

Unless otherwise specified, when a group has one or more linkable sites, any one or more sites of the group can be linked to other groups through chemical bonds. When the linking site of the chemical bond is not positioned, and there is H atom at the linkable site, then the number of H atom at the site will decrease correspondingly with the number of chemical bond linking thereto so as to meet the corresponding valence. The chemical bond between the site and other groups can be represented by a straight solid bond ( [symbol] ), a straight dashed bond ( [symbol] ) or a wavy line ( [symbol] ). For example, the straight solid bond in —OCH₃ means that it is linked to other groups through the oxygen atom in the group; the straight dashed bonds in

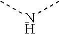

means that it is linked to other groups through the two ends of nitrogen atom in the group; the wave lines in

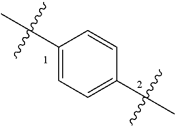 [SMILES: *c1ccc(*)cc1]

means that the phenyl group is linked to other groups through carbon atoms at position 1 and position 2;

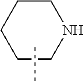

means that it can be linked to other groups through any linkable sites on the piperidinyl by one chemical bond, including at least four types of linkage, including

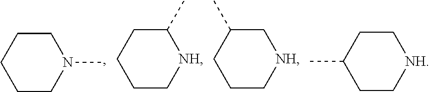

Even though the H atom is drawn on the —N—,

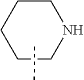

still includes the linkage of

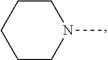

merely when one chemical bond was connected, the H of this site will be reduced by one to the corresponding monovalent piperidinyl.

Unless otherwise specified, the term “C₁₋₃ alkyl” refers to a linear or branched saturated hydrocarbon group consisting of 1 to 3 carbon atoms. The C₁₋₃ alkyl includes C₁₋₂ and C₂₋₃ alkyl and the like; it can be monovalent (such as methyl), divalent (such as methylene) or multivalent (such as methine). Examples of C₁₋₃ alkyl include but are not limited to methyl (Me), ethyl (Et), propyl (including n-propyl and isopropyl), etc.

Unless otherwise specified, Cₙ₋ₙ₊ₘ or C_{n-Cn+m} includes any specific case of n to n+m carbons, for example, C₁₋₁₂ includes C₁, C₂, C₃, C₄, C₅, C₆, C₇, C₈, C₉, C₁₀, C₁₁, and C₁₂, and any range from n to n+m is also included, for example C₁₋₁₂ includes C₁₋₃, C₁₋₆, C₁₋₉, C₃₋₆, C₃₋₉, C₃₋₁₂, C₆₋₉, C₆₋₁₂, and C₉₋₁₂, etc.; similarly, n-membered to n+m-membered means that the number of atoms on the ring is from n to n+m, for example, 3- to 12-membered ring includes 3-membered ring, 4-membered ring, 5-membered ring, 6-membered ring, 7-membered ring, 8-membered ring, 9-membered ring, 10-membered ring, 11-membered ring, and 12-membered ring, and any range from n to n+m is also included, for example, 3- to 12-membered ring includes 3- to 6-membered ring, 3- to 9-membered ring, 5- to 6-membered ring, 5- to 7-membered ring, 6- to 7-membered ring, 6- to 8-membered ring, and 6- to 10-membered ring, etc.

Unless otherwise specified, when double bond structure, such as carbon-carbon double bond, carbon-nitrogen double bond, and nitrogen-nitrogen double bond, exists in the compound, and each of the atoms on the double bond is connected to two different substituents (including the condition where a double bond contains a nitrogen atom, and the lone pair of electrons attached on the nitrogen atom is regarded as a substituent connected), if the atom on the double bond in the compound is connected to its substituent by

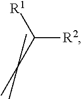

it refers to the (Z) isomer, (E) isomer or a mixture of two isomers of the compound.

The compounds of the present disclosure can be prepared by a variety of synthetic methods known to those skilled in the art, including the specific embodiments listed below, the embodiments formed by their combination with other chemical synthesis methods, and equivalent alternatives known to those skilled in the art, preferred implementations include but are not limited to the embodiments of the present disclosure.

The structure of the compounds of the present disclosure can be confirmed by conventional methods known to those skilled in the art, and if the disclosure involves an absolute configuration of a compound, then the absolute configuration can be confirmed by means of conventional techniques in the art. For example, in the case of single crystal X-ray diffraction (SXRD), the absolute configuration can be confirmed by collecting diffraction intensity data from the cultured single crystal using a Bruker D8 venture diffractometer with CuKα radiation as the light source and scanning mode: φ/ω scan, and after collecting the relevant data, the crystal structure can be further analyzed by direct method (Shelxs97) to confirm the absolute configuration.

The solvent used in the present disclosure is commercially available.

The present disclosure adopts the following abbreviations: eq stands for equivalent; Pd₂(dba)₃ stands for tris(dibenzylideneacetone)dipalladium; Xantphos stands for 4,5-bis(diphenylphosphino)-9,9-dimethylxanthene; BAK stands for Bcl-2 homologous antagonist; BAD stands for Bcl-2 associated cell death agonist; Noxa stands for phorbol-12-myristate-13-acetate-induced protein; GST stands for glutathione-S-transferase; HTRF stands for homogeneous time-resolved fluorescence; FAM stands for fluorescein labeled; EDTA stands for ethylene diamine tetraacetic acid; Tritonx-100 stands for Triton X-100; DMSO stands for dimethyl sulfoxide; CD₃OD stands for deuterated methanol; prep-HPLC stands for high performance liquid phase preparation; RBC stands for Reaction Biology Corporation; ATP stands for adenosine triphosphate; MCL stands for myeloid leukemia; ABT stands for AbbVie; RS4;11 stands for an acute lymphoblastic leukemia tumor cell line; CTG stands for luminescent live cell detection system; Bn stands for benzyl; SEM stands for 2-(trimethylsilyl)ethoxymethyl; ACN stands for acetonitrile; CO₂ stands for carbon dioxide; NADPH stands for nicotinamide adenine dinucleotide phosphate; RFU stands for measured fluorescence.

The compounds of the present disclosure are named according to the conventional naming principles in the art or by ChemDraw® software, and the commercially available compounds use the supplier catalog names.

### DETAILED DESCRIPTION OF THE PREFERRED EMBODIMENT

The present disclosure is described in detail by the embodiments below, but it does not mean that there are any adverse restrictions on the present disclosure. The present disclosure has been described in detail herein, wherein specific embodiments thereof are also disclosed, and it will be apparent to those skilled in the art that various variations and improvements can be made to specific embodiments of the present disclosure without departing from the spirit and scope of the present disclosure.

### Embodiment 1

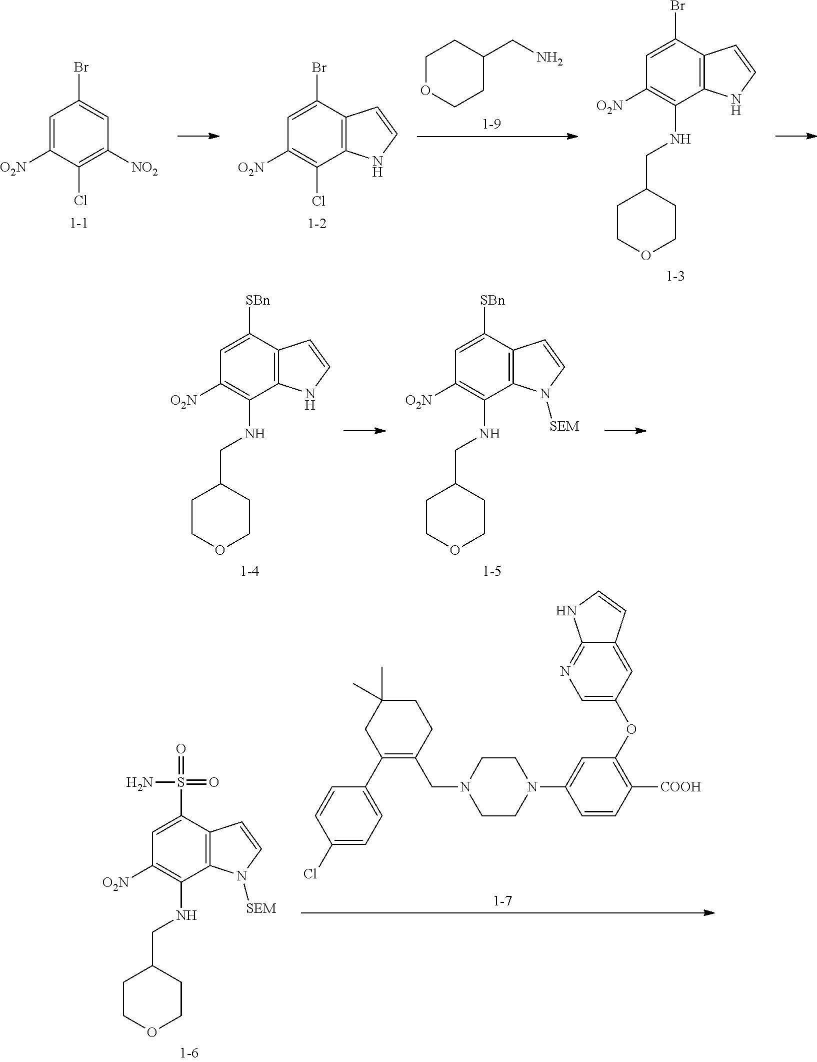

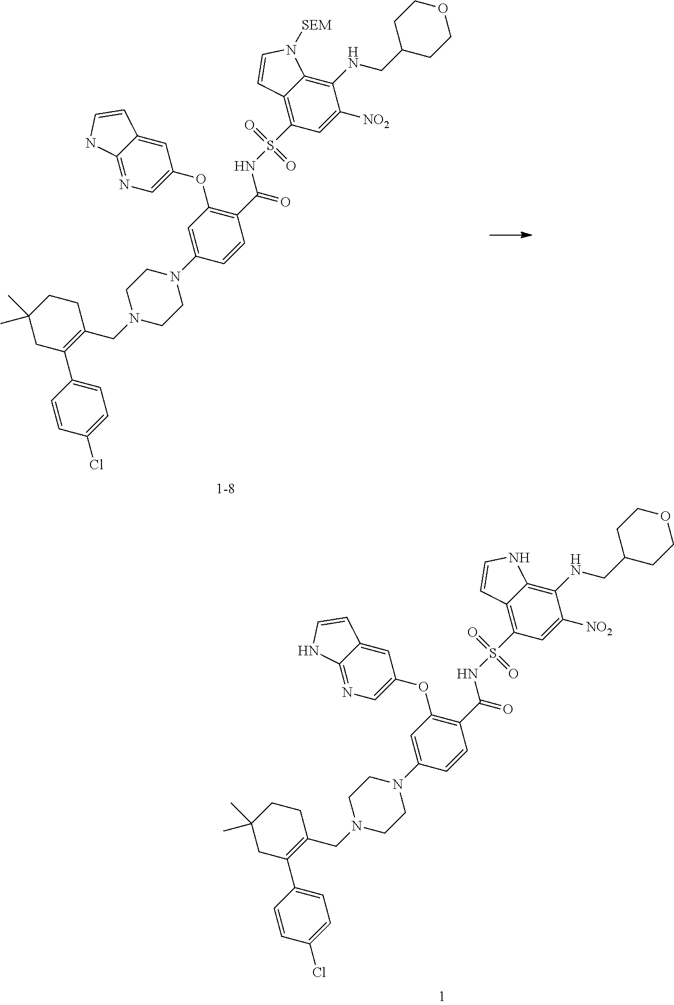 [SMILES: CC1(C)CCC(CN2CCN(c3ccc(C(=O)NS(=O)(=O)c4cc([N+](=O)[O-])c(NCC5CCOCC5)c5c4ccn5COCC[Si](C)(C)C)c(Oc4cnc5[nH]ccc5c4)c3)CC2)=C(c2ccc(Cl)cc2)C1 ; CC1(C)CCC(CN2CCN(c3ccc(C(=O)NS(=O)(=O)c4cc([N+](=O)[O-])c(NCC5CCOCC5)c5[nH]ccc45)c(Oc4cnc5[nH]ccc5c4)c3)CC2)=C(c2ccc(Cl)cc2)C1]

### Step 1: Synthesis of Compound 1-2

At −78° C., vinylmagnesium bromide (24.8 mL, 1 M) was slowly added to the tetrahydrofuran (20 mL) solution of compound 1-1 (2 g, 7.11 mmol). The reaction solution was stirred at −40° C. for 3 hours after the dropwise addition was completed. After the reaction was completed, the mixture was poured into saturated aqueous ammonium chloride solution (50 mL), stirred at 25° C. for 10 minutes, extracted with ethyl acetate (50 mL×3 times), and the organic phase was washed with saturated brine (30 mL), dried over anhydrous sodium sulfate, and concentrated under reduced pressure to obtain a crude product, and the crude product was purified by silica gel column chromatography (petroleum ether/ethyl acetate=50/1 to 3/1) to obtain compound 1-2.

¹H NMR (400 MHz, DMSO-d₆) δ ppm 6.60-6.71 (m, 1H), 7.91 (t, J=2.87 Hz, 1H), 7.97-8.07 (m, 1H), 12.65 (br s, 1H).

### Step 2: Synthesis of Compound 1-3

Potassium carbonate (360.21 mg, 2.61 mmol) and compound 1-9 (300.18 mg, 2.61 mmol) were added to the DMSO (44 mL) solution of compound 1-2 (359 mg, 1.30 mmol), and the reaction solution was stirred at 110° C. for 16 hours. After the reaction was completed, the mixture was poured into water (80 mL), extracted with ethyl acetate (50 mL×3 times), and the organic phase was washed with saturated brine (30 mL), dried over anhydrous sodium sulfate, and concentrated under reduced pressure to obtain a residue, and the residue was purified by silica gel column chromatography (petroleum ether/ethyl acetate=5/1 to 1/1) to obtain compound 1-3.

¹H NMR (400 MHz, DMSO-d₆) δ ppm 1.31 (qd, J=12.26, 4.38 Hz, 2H), 1.67 (br dd, J=12.76, 1.50 Hz, 2H), 1.90 (ddt, J=11.02, 7.24, 3.75, 3.75 Hz, 1H), 3.33 (s, 2H), 3.74 (t, J=6.13 Hz, 2H), 3.87 (br dd, J=11.38, 3.00 Hz, 2H), 6.45-6.51 (m, 1H), 7.66 (t, J=2.81 Hz, 1H), 7.87 (s, 1H), 9.06 (br t, J=5.19 Hz, 1H), 11.71-12.05 (m, 1H).

### Step 3: Synthesis of Compound 1-4

A mixture of compound 1-3 (410 mg, 1.16 mmol), benzyl mercaptan (215.66 mg, 1.74 mmol), N,N-diisopropylethylamine (448.80 mg, 3.47 mmol), Pd₂(dba)₃ (106.0 mg, 0.12 mmol) and Xantphos (133.96 mg, 0.23 mmol) in toluene (4 mL) was replaced with nitrogen for three times, then the mixture was stirred at 110° C. for 16 hous under nitrogen atmosphere. The reaction mixture was poured into water (20 mL), extracted with ethyl acetate (30 mL×3). The organic phase was washed with saturated brine (30 mL), dried over anhydrous sodium sulfate, and concentrated under reduced pressure to obtain a residue, and the residue was purified by silica gel column chromatography (petroleum ether/ethyl acetate=4/1 to 1/2) to obtain compound 1-4.

¹H NMR (400 MHz, DMSO-d₆) δ=11.80-11.64 (m, 1H), 9.13 (t, J=5.3 Hz, 1H), 7.64-7.63 (m, 1H), 7.62 (d, J=2.8 Hz, 1H), 7.28 (br d, J=2.9 Hz, 5H), 6.60-6.58 (m, 1H), 4.14-4.13 (m, 2H), 3.90-3.85 (m, 2H), 3.76-3.73 (m, 2H), 3.32-3.29 (m, 2H), 1.92-1.88 (m, 1H), 1.69-1.65 (m, 2H), 1.32 (br dd, J=3.8, 12.4 Hz, 2H).

### Step 4: Synthesis of Compound 1-5

At 0° C., sodium hydride (44.28 mg, 1.11 mmol, 60% purity) was addded to the N,N-dimethylformamide (2 mL) solution of compound 1-4 (200 mg, 0.50 mmol) and the mixture was stirred for 0.5 hours. 2-(Trimethylsilyl)ethoxymethyl chloride (100.66 mg, 603.79 μmol, 106.86 μL) was then added dropwise at 0° C., and the mixture was stirred at 0° C. for 3 hours, then heated to 28° C. The mixture was stirred for another 12 hours. The mixture was diluted with water (100 mL) and then extracted with ethyl acetate (100 mL×2). The organic phase was washed with saturated ammonium chloride solution (40 mL) and saturated brine (30 mL), dried over anhydrous sodium sulfate and concentrated under reduced pressure. The crude product was purified by silica gel column chromatography (petroleum ether/ethyl acetate=25/1 to 1/1) to obtain compound 1-5.

MS (ESI) m/z: 528 [M+H]⁺.

### Step 5: Synthesis of Compound 1-6

At 0° C., N-chlorosuccinimide (15.18 mg, 113.69 μmol) was added to the acetonitrile (1 mL), acetic acid (0.2 mL) and water (0.4 mL) solution of compound 1-5 (20 mg, 37.90 μmol) in batches and the mixture was stirred at 0° C. for 2 hours. Additional N-chlorosuccinimide (0.2 g) was added thereto at 20° C. and stirred for 1 hour. At 0° C., the reaction mixture was added dropwise to ammonia water (2.3 mL, 25% purity), stirred for 1 hour, then diluted with water (10 mL) and extracted with a mixed solvent (ethyl acetate/ethanol=5/1, 15 mL×3). The combined organic phase was washed with brine, dried over anhydrous sodium sulfate, filtered, and concentrated under reduced pressure to obtain a residue. Ethyl acetate (3 mL) was added to the residue and the mixture was slurried at 20° C. for 1 hour, filtered, and the filter cake was collected and dried under vacuum to obtain compound 1-6.

MS (ESI) m/z: 485 [M+H]⁺.

### Step 6: Synthesis of Compound 1-8

Compound 1-7 (25.93 mg, 45.39 μmol) and triethylamine (8.35 mg, 82.54 μmol) were added to the dichloromethane (2 mL) solution of compound 1-6 (20 mg, 41.27 μmol), 1-(3-dimethylaminopropyl)-3-ethylcarbodiimide hydrochloride (9.49 mg, 49.52 μmol) and 4-dimethylaminopyridine (10.08 mg, 82.54 μmol). The mixture was stirred at 40° C. for 16 hours. The mixture was concentrated under reduced pressure, and purified by silica gel column chromatography (dichloromethane/methanol=50/1 to 10/1) to obtain compound 1-8.

### Step 7: Synthesis of Compound 1

Trifluoroacetic acid (0.3 mL) was added to the dichloromethane (0.3 mL) solution of compound 1-8 (30 mg, 19 μmol). Then the mixture was stirred at 20° C. for 16 hours. The mixture was then concentrated under reduced pressure and dissolved in methanol (0.6 mL), and then potassium carbonate (5.3 mg, 38 μmol) was added thereto, and the mixture was then stirred at 28° C. for 1 hour. The mixture was diluted with dichloromethane/methanol (10/1, 60 mL). Then the mixture was washed with saturated ammonium chloride (15 mL) and saturated brine (10 mL), dried over anhydrous sodium sulfate and concentrated under reduced pressure. The crude product was purified by prep_HPLC (trifluoroacetic acid system) (chromatographic column: Phenomenex luna C18 150*25 mm*10 μm; mobile phase: [water (0.225% trifluoroacetic acid)-acetonitrile]; B (acetonitrile) %: 40% to 70%, 8 min) to obtain compound 1 (trifluoroacetate).

¹H NMR (400 MHz, DMSO-d₆) δ ppm 0.91 (s, 6H), 1.35-1.39 (m, 2H), 1.41-1.49 (m, 2H), 1.68 (br d, J=11.86 Hz, 2H), 1.97-2.02 (m, 4H), 2.06 (s, 1H), 2.14 (br d, J=4.16 Hz, 6H), 2.69-2.71 (m, 1H), 2.99 (br s, 2H), 3.67-3.79 (m, 4H), 3.79-3.96 (m, 4H), 5.28-5.39 (m, 2H), 6.08-6.19 (m, 1H), 6.34-6.45 (m, 1H), 6.60-6.65 (m, 1H), 6.77-6.85 (m, 1H), 7.01-7.05 (m, 2H), 7.16-7.25 (m, 1H), 7.31-7.35 (m, 2H), 7.50 (br s, 2H), 7.94-8.03 (m, 1H), 8.25-8.47 (m, 1H), 9.18-9.26 (m, 1H), 11.60-11.72 (m, 1H).

### Embodiment 2

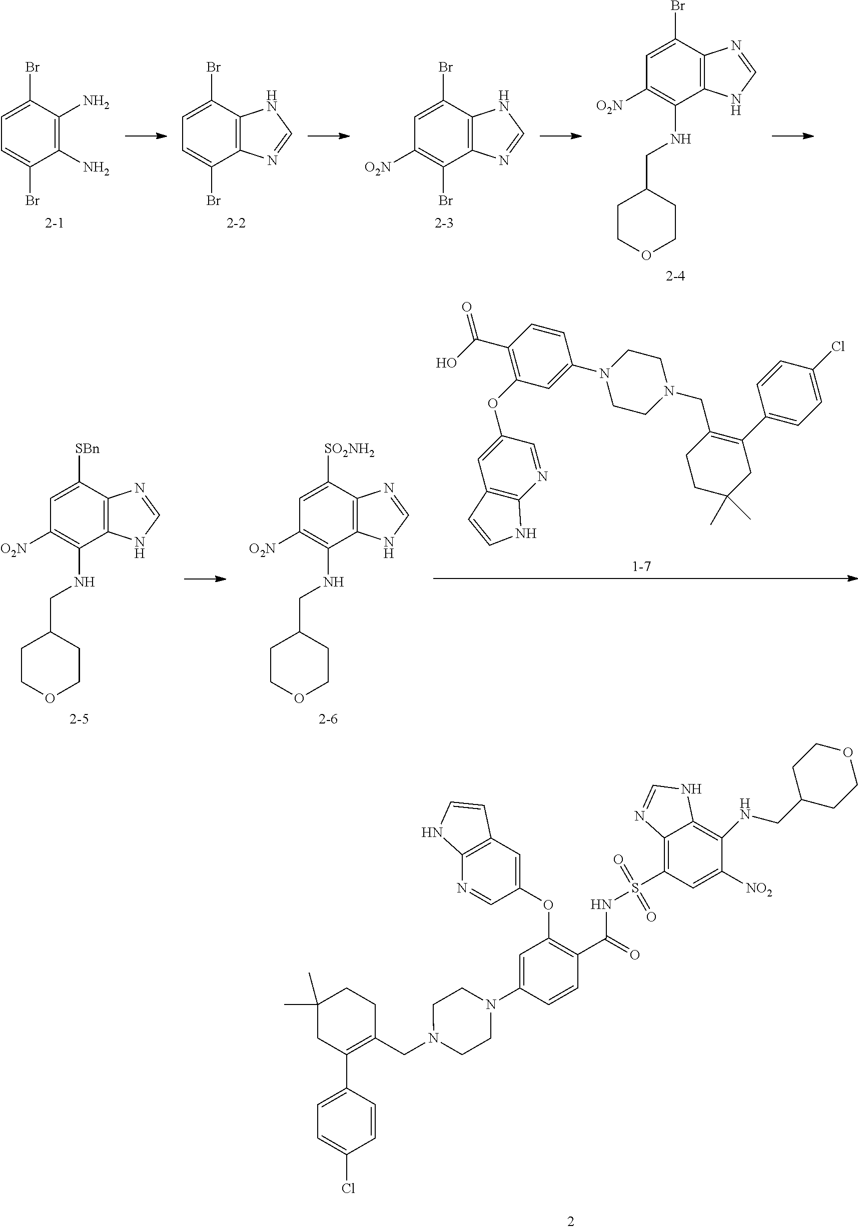

### Step 1: Synthesis of Compound 2-2

Trimethyl orthoformate (1.98 g, 18.61 mmol, 2.04 mL) and p-toluenesulfonic acid (213.69 mg, 1.24 mmol) were added to the tetrahydrofuran (40 mL) solution of compound 2-1 (3.3 g, 12.41 mmol). The mixture was stirred at 25° C. for 10 minutes, then filtered, and the filter cake was collected and dried under vacuum to obtain compound 2-2.

¹H NMR (400 MHz, DMSO-d₆) δ=13.23 (br s, 1H), 8.41 (s, 1H), 7.39 (s, 2H).

### Step 2: Synthesis of Compound 2-3

Potassium nitrate (87.94 mg, 869.79 μmol) was added to the sulfuric acid (1.2 mL, 98%) solution of compound 2-2 (200 mg, 724.83 μmol) at 0° C. The mixture was stirred at 0° C. for 1 hour. The mixture was poured into the mixture of ice water (5 mL) and ammonia water (5 mL), filtered, and the filter cake was collected and dried under vacuum to obtain compound 2-3.

¹H NMR (400 MHz, DMSO-d₆) δ=13.46 (br s, 1H), 8.63 (s, 1H), 8.17 (s, 1H).

### Step 3: Synthesis of Compound 2-4

Potassium carbonate (2.33 g, 16.83 mmol) and (tetrahydro-2H-pyran-4-yl)methylamine (1.94 g, 16.83 mmol) were added to the N,N-dimethylformamide (18 mL) solution of compound 2-3 (1.8 g, 5.61 mmol). The mixture was stirred at 120° C. for 16 hours. The reaction mixture was poured into water (200 mL) to precipitate the solid, filtered, and the filter cake was collected and dried under vacuum to obtain compound 2-4.

¹H NMR (400 MHz, CDCl₃) δ=9.27 (br s, 2H), 8.27 (s, 1H), 7.87 (s, 1H), 4.13 (t, J=6.5 Hz, 2H), 3.94 (br dd, J=3.8, 10.9 Hz, 2H), 3.33 (dt, J=2.0, 11.8 Hz, 2H), 1.96-1.83 (m, 1H), 1.71 (br d, J=12.5 Hz, 2H), 1.44-1.35 (m, 1H), 1.34 (br s, 1H).

### Step 4: Synthesis of Compound 2-5

The toluene (7 mL) solution of compound 2-4 (700 mg, 1.97 mmol, 1 eq), benzyl mercaptan (489.56 mg, 3.94 mmol, 461.85 μL, 2 eq), N,N-diisopropylethylamine (764.13 mg, 5.91 mmol, 1 mL), Pd₂(dba)₃ (180.47 mg, 197.08 μmol) and Xantphos (228.07 mg, 394.16 μmol) was replaced with nitrogen for three times, then the reaction sloution was stirred at 110° C. for 16 hous under nitrogenatmosphere. The reaction mixture was poured into water (20 mL), extracted with ethyl acetate (20 mL×3). The organic phase was washed with saturated brine (30 mL), dried over anhydrous sodium sulfate, and concentrated under reduced pressure to obtain a residue, and the residue was purified by silica gel column chromatography (petroleum ether/ethyl acetate=20/1 to 3/1) to obtain compound 2-5.

¹H NMR (400 MHz, CDCl₃) δ=9.39 (br s, 1H), 8.82 (br s, 1H), 8.34 (s, 1H), 7.60 (s, 1H), 7.22 (dd, J=1.9, 4.9 Hz, 3H), 7.12-7.06 (m, 2H), 4.22 (t, J=6.5 Hz, 2H), 4.03 (dd, J=3.3, 11.0 Hz, 2H), 3.92 (s, 2H), 3.42 (dt, J=2.0, 11.8 Hz, 2H), 2.05-1.93 (m, 1H), 1.80 (br dd, J=1.8, 12.9 Hz, 2H), 1.54-1.42 (m, 2H).

### Step 5: Synthesis of Compound 2-6

At 0° C., N-chlorosuccinimide (854.52 mg, 6.40 mmol) was added to the acetonitrile (32 mL), acetic acid (0.4 mL) and water (0.8 mL) solution of compound 2-5 (850 mg, 2.13 mmol, 1 eq) in batches and the mixture was stirred at 0° C. for 2 hours, then additional N-chlorosuccinicimide (0.2 g) was added thereto at 20° C. and stirred for 1 hour. At 0° C., the reaction mixture was added dropwise to ammonia water (27.30 g, 194.75 mmol, 30.00 mL, 25% purity) and stirred for 1 hour. The reaction mixture was diluted with water (20 mL) and extracted with the mixed solvent (ethyl acetate/ethanol=5/1, 50 mL×4). The combined organic phase was washed with brine (20 mL×2), dried over sodium sulfate, filtered, and concentrated under reduced pressure to obtain a residue. The residue was added with ethyl acetate (10 mL) and stirred at 20° C. for 1 hour, filtered, and the filter cake was collected and dried under vacuum to obtain compound 2-6.

¹H NMR (400 MHz, DMSO-d₆) δ=11.43 (br s, 2H), 9.40 (t, J=6.3 Hz, 1H), 8.38 (s, 1H), 8.28-8.13 (m, 1H), 6.13 (br s, 1H), 4.27 (t, J=6.6 Hz, 2H), 3.85 (br dd, J=3.1, 11.3 Hz, 2H), 3.26-3.23 (m, 2H), 2.01-1.87 (m, 1H), 1.64 (br d, J=11.2 Hz, 2H), 1.37-1.22 (m, 2H).

### Step 6: Synthesis of Compound 2

Compound 1-7 (48.21 mg, 84.42 μmol) and triethylamine (17.08 mg, 168.84 23.50 μL) were added to the dichloromethane (2 mL) solution of compound 2-6 (30 mg, 84.42 μmol), 1-(3-dimethylaminopropyl)-3-ethylcarbodiimide hydrochloride (19.42 mg, 101.30 μmol) and 4-dimethylaminopyridine (20.63 mg, 168.84 μmol). The mixture was stirred at 40° C. for 16 hours. The mixture was concentrated under reduced pressure, and purified by prep_HPLC (acidic system) (chromatographic column: Unisil 3-100 C18 Ultra 150*50 mm*3 μm; mobile phase: [water (0.225% trifluoroacetic acid)-acetonitrile]; B (acetonitrile) %: 40% to 60%, 10 min) to obtain compound 2 (trifluoroacetate).

¹H NMR (400 MHz, DMSO-d₆) δ=11.63 (br s, 1H), 9.37 (br s, 1H), 8.43 (br s, 1H), 8.20-7.91 (m, 2H), 7.66-7.22 (m, 6H), 7.04 (br d, J=7.5 Hz, 2H), 6.63 (br d, J=7.5 Hz, 2H), 6.36 (br s, 1H), 6.17 (br s, 1H), 4.22 (br s, 2H), 3.84 (br d, J=8.9 Hz, 2H), 3.22-3.17 (m, 2H), 3.03 (br s, 3H), 2.74 (br s, 2H), 2.19 (br s, 6H), 1.95 (br s, 3H), 1.64 (br d, J=10.8 Hz, 2H), 1.41-1.20 (m, 5H), 0.92 (br s, 6H).

### Embodiment 3

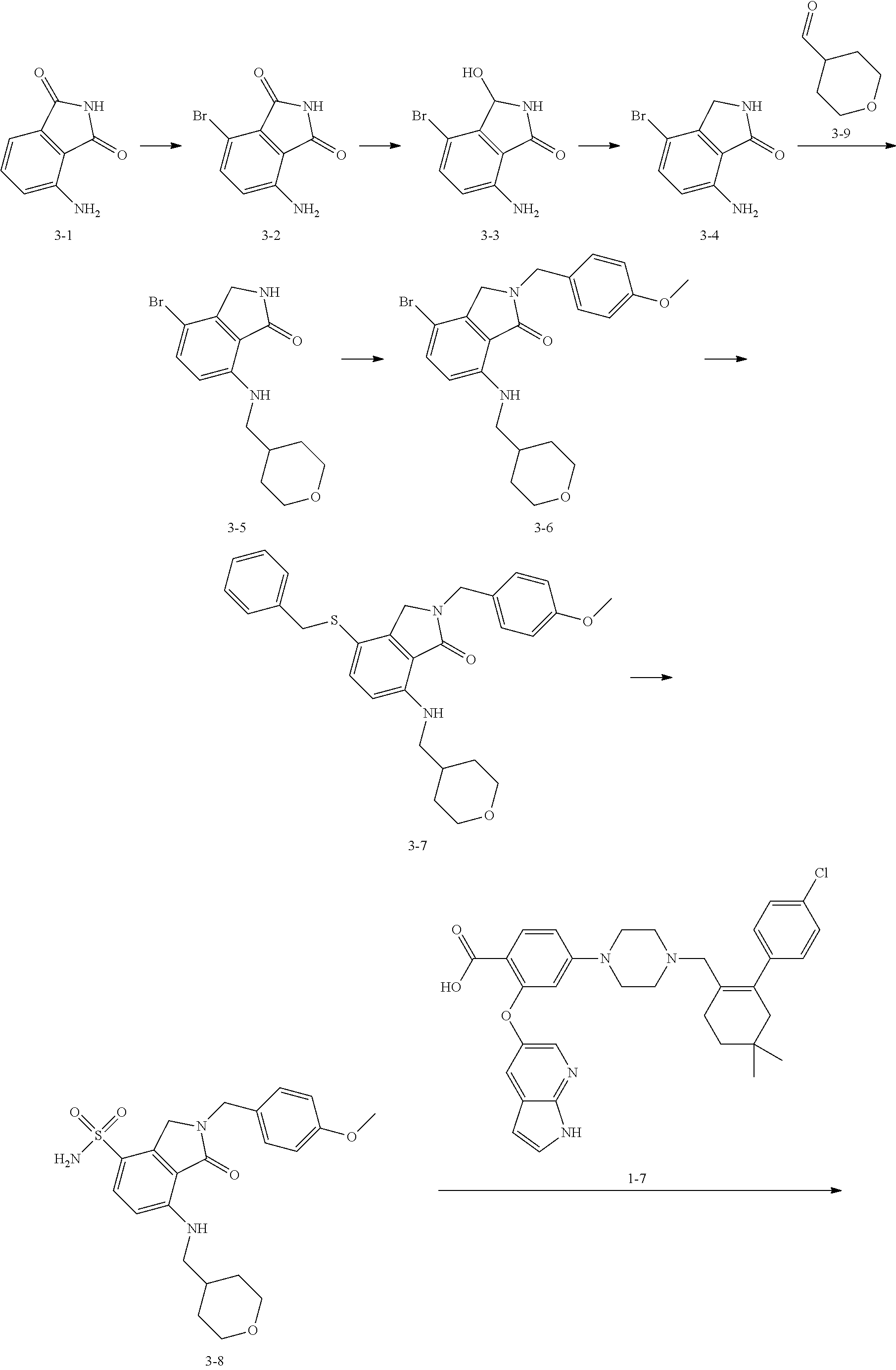

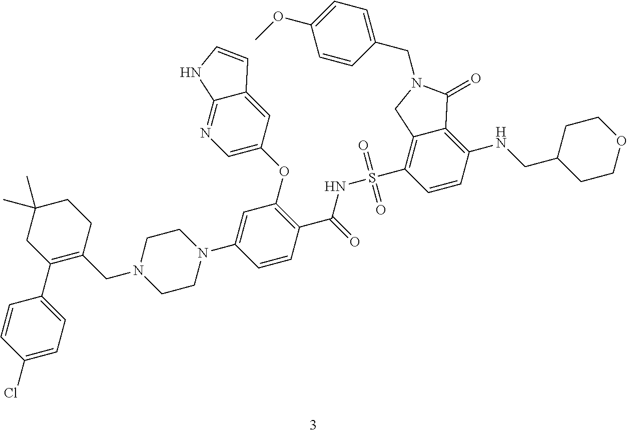 [SMILES: COc1ccc(CN2Cc3c(S(=O)(=O)NC(=O)c4ccc(N5CCN(CC6=C(c7ccc(Cl)cc7)CC(C)(C)CC6)CC5)cc4Oc4cnc5[nH]ccc5c4)ccc(NCC4CCOCC4)c3C2=O)cc1]

### Step 1: Synthesis of Compound 3-2

N-Bromosuccinimide (5.49 g, 30.84 mmol) was added to the methanol (200 mL) solution of compound 3-1 (5 g, 30.84 mmol) and the mixture was stirred at 28° C. for 1 hour. The reaction solution was filtered, and the filter cake was washed with methanol for three times and the filtrate was combined and concentrated under reduced pressure to obtain compound 3-2.

¹H NMR (400 MHz, DMSO-d₆) δ=11.10 (s, 1H), 7.51 (d, J=8.8 Hz, 1H), 6.90 (d, J=8.8 Hz, 1H), 6.56 (br s, 2H).

### Step 2: Synthesis of Compound 3-3

Diisobutyl aluminum hydride (1 M, 72.60 mL) was added dropwise to the tetrahydrofuran (300 mL) solution of compound 3-2 (3.5 g, 14.52 mmol) at −78° C. and stirred at 28° C. for 16 hours. The reaction solution was poured into 50 mL of water, extracted with ethyl acetate (150 mL×3), washed with saturated brine (100 mL), dried over anhydrous sodium sulfate, and concentrated under reduced pressure to obtain compound 3-3.

LCMS (ESI) m/z: 243/245[M+H]⁺.

### Step 3: Synthesis of Compound 3-4

At 0° C., triethylsilane (3.35 g, 28.8 mmol) was added to the trifluoroacetic acid (20 mL) solution of compound 3-3 (3.5 g, 14.4 mmol), and the mixture was stirred at 0° C. for 2 hours, and then the reaction solution was poured into 20 mL of saturated sodium bicarbonate solution, stirred at room temperature for 0.5 hours, then filtered, and the filter cake was collected and dried under vacuum to obtain compound 3-4.

¹H NMR (400 MHz, DMSO-d₆) δ=8.34 (s, 1H), 7.29 (d, J=8.6 Hz, 1H), 6.52 (d, J=8.5 Hz, 1H), 6.16 (s, 2H), 4.10 (s, 2H).

### Step 4: Synthesis of Compound 3-5

Sodium triacetoxyborohydride (2.99 g, 14.09 mmol) and acetic acid (2.42 mL) were slowly added to the dichloroethane (30 mL) solution of compound 3-4 (1.6 g, 2.05 mmol) and compound 3-9 (1.45 g, 12.68 mmol). The mixture was stirred at 25° C. for 16 hours. The reaction solution was poured into 30 mL of water, extracted with dichloromethane (30 mL×3), washed with saturated brine (20 mL), and finally dried over anhydrous sodium sulfate. The organic phase was concentrated to obtain a crude product, and the crude product was purified by silica gel column chromatography (petroleum ether/ethyl acetate=25/1 to 5/1) to obtain compound 3-5.

¹H NMR (400 MHz, DMSO-d₆) δ=8.49 (s, 1H), 7.42 (d, J=8.8 Hz, 1H), 6.84 (t, J=6.1 Hz, 1H), 6.61 (d, J=8.8 Hz, 1H), 4.16 (s, 2H), 3.85 (br dd, J=3.0, 11.3 Hz, 2H), 3.27 (dt, J=1.9, 11.7 Hz, 2H), 3.10 (t, J=6.5 Hz, 2H), 1.86-1.73 (m, 1H), 1.60 (br dd, J=1.8, 12.7 Hz, 2H), 1.23 (dq, J=4.6, 12.3 Hz, 2H).

### Step 5: Synthesis of Compound 3-6

At 0° C., sodium hydride (525.30 mg, 13.13 mmol, 60% purity) was added to the tetrahydrofuran (15 mL) solution of compound 3-5 (0.76 g, 2.34 mmol) in batches and the mixture was stirred for 0.5 hours; then p-methoxybenzyl bromide (0.8 g, 5.1 mmol) was added thereto, and the reaction solution was stirred at 25° C. for 16 hours and continued to stir at 55° C. for 16 hours. The reaction solution was poured into 20 mL of water, extracted with ethyl acetate (50 mL×3), washed with saturated brine (30 mL), and finally dried over anhydrous sodium sulfate. The organic phase was concentrated to obtain a crude product, and the crude product was subjected to silica gel column chromatography (petroleum ether/ethyl acetate=2/1 to 1/2) to obtain compound 3-6.

LCMS (ESI) m/z: 445/447[M+H]⁺.

### Step 6: Synthesis of Compound 3-7

A mixture of compound 3-6 (200.00 mg, 449.09 μmol), benzyl mercaptan (83.67 mg, 673.63 μmol), Pd₂(dba)₃ (41.12 mg, 44.91 μmol), Xantphos (51.97 mg, 89.82 μmol) and diisopropylethylamine (174.12 mg, 1.35 mmol) in toluene (4 mL) was replaced with nitrogen for three times, then the mixture was stirred at 110° C. for 16 hours under nitrogen atmosphere. The reaction mixture was poured into water (10 mL), extracted with ethyl acetate (10 mL×3). The organic phase was washed with saturated brine (20 mL), dried over anhydrous sodium sulfate, filtered and the filtrate was concentrated under reduced pressure, and the residue was purified by silica gel column chromatography (petroleum ether/ethyl acetate=1/0 to 5/1) to obtain compound 3-7.

MS (ESI) m/z: 489 [M+H]⁺.

### Step 7: Synthesis of Compound 3-8

At 0° C., N-chlorosuccinimide (122.97 mg, 920.92 μmol) was added to a mixture of compound 3-7 (150 mg, 306.97 μmol), acetonitrile (3 mL), acetic acid (0.3 mL) and water (0.6 mL) in batches. The mixture was stirred at 25° C. for 32 hours. Then, the reaction mixture was added to ammonia water (5.46 g, 38.94 mmol, 6.00 mL, 25% purity) and stirred at 25° C. for 2 hours. The reaction mixture was diluted with water (30 mL), extracted with ethyl acetate (30 mL×3). The combined organic phase was washed with saturated brine (30 mL), dried over anhydrous sodium sulfate, filtered, and the filtrate was concentrated under reduced pressure to obtain a residue, and the residue was purified by thin layer chromatography (silica gel, petroleum ether/ethyl acetate=1/2) to obtain compound 3-8.

MS (ESI) m/z: 446 [M+H]⁺.

### Step 8: Synthesis of Compound 3

Compound 3-8 (0.03 g, 0.067 mmol), compound 1-7 (0.038 g, 0.067 mmol), 4-dimethylaminopyridine (0.016 g, 0.13 mmol), 1-(3-dimethylaminopropyl)-3-ethylcarbodiimide hydrochloride (0.016 g, 0.08 mmol) and triethylamine (0.014 g, 0.13 mmol) were added to dichloromethane (10 mL), and the mixture was stirred at 30° C. for 16 hours. The reaction solution was poured into 20 mL of water, extracted with dichloromethane (20 mL×3), washed with saturated brine (20 mL×2), and finally dried over anhydrous sodium sulfate. The organic phase was concentrated to obtain a crude product, and the crude product was separated by prep-HPLC (chromatographic column: Phenomenex Gemini-NX C18 75*30 mm*3 μm; mobile phase: [water (0.1% trifluoroacetic acid)-acetonitrile]; B (acetonitrile)%: 52% to 62%, 7 minutes) to obtain compound 3 (trifluoroacetate).

¹H NMR (400 MHz, DMSO-d₆) δ=11.79 (s, 1H), 11.57 (br s, 1H), 9.43 (br s, 1H), 8.07 (d, J=2.6 Hz, 1H), 7.65 (d, J=8.9 Hz, 1H), 7.62-7.51 (m, 3H), 7.45 (d, J=8.8 Hz, 1H), 7.40 (d, J=8.4 Hz, 2H), 7.10 (t, J=8.6 Hz, 4H), 6.94-6.81 (m, 2H), 6.75-6.62 (m, 2H), 6.44 (dd, J=1.9, 3.4 Hz, 1H), 6.22 (d, J=2.0 Hz, 1H), 4.43 (s, 3H), 3.91-3.81 (m, 2H), 3.72 (s, 3H), 3.68-3.51 (m, 4H), 3.28 (br dd, J=10.1, 11.5 Hz, 3H), 3.21-3.15 (m, 2H), 3.03 (br s, 2H), 2.74 (br s, 2H), 2.20 (br s, 2H), 2.02 (br s, 2H), 1.82 (dtt, J=3.6, 7.1, 11.0 Hz, 1H), 1.68-1.56 (m, 2H), 1.45 (br t, J=6.1 Hz, 2H), 1.34-1.19 (m, 3H), 0.94 (s, 6H). MS (ESI) m/z: 998 [M+H]⁺.

### Embodiment 4

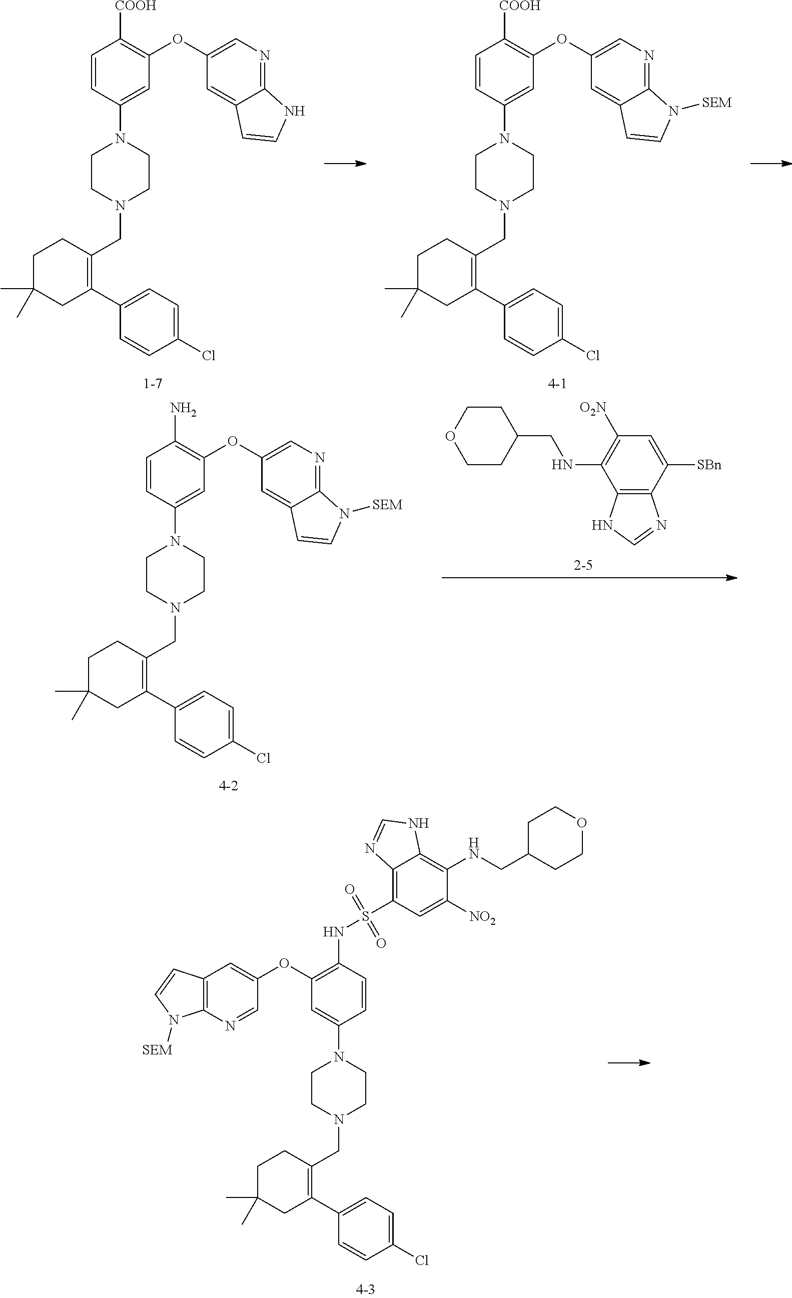

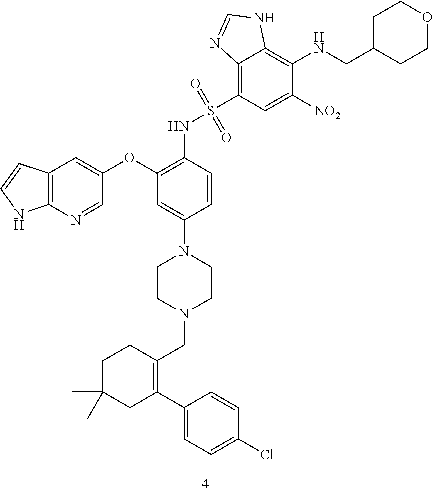 [SMILES: CC1(C)CCC(CN2CCN(c3ccc(NS(=O)(=O)c4cc([N+](=O)[O-])c(NCC5CCOCC5)c5[nH]cnc45)c(Oc4cnc5[nH]ccc5c4)c3)CC2)=C(c2ccc(Cl)cc2)C1]

### Step 1: Synthesis of Compound 4-1

At 0° C., sodium hydride (525.30 mg, 13.13 mmol, 60% purity) was addded to the N,N-dimethylformamide (30 mL) solution of compound 1-7 (3 g, 5.25 mmol) in batches and the mixture was stirred for 0.5 hours. 2-(Trimethylsilyl)ethoxymethyl chloride (1.84 g, 11.03 mmol, 1.95 mL) was added dropwise at 0° C., and the mixture was stirred at 0° C. for 3 hours, then heated to 28° C. The mixture was stirred for another 12 hours. The mixture was diluted with water (100 mL) and then extracted with ethyl acetate (100 mL×2). The organic phase was washed with saturated ammonium chloride solution (40 mL) and saturated brine (30 mL), dried over anhydrous sodium sulfate and concentrated under reduced pressure. The crude product was purified by silica gel column chromatography (petroleum ether/ethyl acetate=10/1 to 1/1) to obtain compound 4-1.

MS (ESI) m/z: 701 [M+H]⁺.

### Step 2: Synthesis of Compound 4-2

Triethylamine (173.13 mg, 1.71 mmol, 238.14 μL) and diphenyl azidophosphate (204.04 mg, 741.41 μmol, 160.66 μL) were added to the toluene (8 mL) solution of compound 4-1 (400 mg, 570.31 μmol). Then the mixture was stirred at 45° C. for 12 hours. Then ethanol (131.37 mg, 2.85 mmol, 166.71 μL) was added and the mixture was stirred at 70° C. for 3 hours, then potassium hydroxide (319.98 mg, 5.70 mmol) and ethanol (2.5 mL) were added and the reaction sloution was stirred at 90° C. for 12 hours. The mixture was diluted with ethyl acetate (30 mL), and then washed with saturated ammonium chloride solution (15 mL) and saturated brine (10 mL), dried over anhydrous sodium sulfate and concentrated under vacuum. The residue was purified by silica gel column chromatography (petroleum ether/ethyl acetate=12/1 to 6/1) to obtain compound 4-2.

MS (ESI) m/z: 672 [M+H]⁺.

### Step 3: Synthesis of Compound 4-3

At 0° C., N-chlorosuccinimide (58.64 mg, 439.17 μmol) was added to the acetonitrile (0.80 mL), water (0.02 mL) and acetic acid (0.01 mL) solution of compound 2-5 (56.82 mg, 125.48 μmol) in batches and the mixture was stirred for 1 hour. The mixture was then heated to 28° C. and stirred for another 2 hours. The mixture was then added dropwise to the stirred acetonitrile (1.6 mL) solution of compound 4-2 (75.93 mg, 112.93 μmol) and pyridine (49.63 mg, 627.39 μmol, 50.64 μL). The mixture was stirred at 28° C. for 12 hours. The mixture was concentrated under reduced pressure, dissolved in dichloromethane (20 mL) and ethyl acetate (20 mL), washed with saturated ammonium chloride (10 mL) and saturated brine (10 mL), dried over anhydrous sodium sulfate and concentrated under reduced pressure. The crude product was purified by thin layer chromatography plate (silica gel, petroleum ether/ethyl acetate/dichloromethane=1/7/1.5) to obtain compound 4-3.

MS (ESI) m/z: 1010 [M+H]⁺.

### Step 4: Synthesis of Compound 4

Trifluoroacetic acid (0.3 mL) was added to the dichloromethane (0.3 mL) solution of compound 4-3 (30 mg, 29.68 μmol). Then the mixture was stirred at 28° C. for 8 hours. The mixture was then concentrated under reduced pressure and dissolved in methanol (0.6 mL), and then potassium carbonate (20.51 mg, 148.41 μmol) was added thereto, and the mixture was then stirred at 28° C. for 1 hour. The mixture was diluted with dichloromethane/methanol (10/1, 30 mL). Then the mixture was washed with saturated ammonium chloride (10 mL×2) and saturated brine (10 mL), dried over anhydrouxs sodium sulfate and concentrated under reduced pressure. The residue was purified by prep_HPLC (trifluoroacetic acid system) (chromatographic column: Phenomenex luna C18 150*25 mm*10 μm; mobile phase: [water (0.1% trifluoroacetic acid-acetonitrile]; B (acetonitrile) %: 39% to 69%, 10 min) to obtain compound 4 (trifluoroacetate).

¹H NMR (400 MHz, CD₃OD) δ=8.11 (s, 1H), 7.78 (s, 1H), 7.42 (d, J=8.9 Hz, 1H), 7.30 (d, J=3.4 Hz, 1H), 7.28-7.21 (m, 2H), 6.95 (d, J=8.4 Hz, 2H), 6.67-6.59 (m, 2H), 6.15 (d, J=3.4 Hz, 2H), 6.04 (d, J=2.3 Hz, 1H), 3.91-3.80 (m, 4H), 3.53 (s, 3H), 3.35-3.25 (m, 4H), 2.89-2.58 (m, 4H), 2.12 (br t, J=6.0 Hz, 2H), 1.99 (br s, 2H), 1.88-1.77 (m, 1H), 1.64-1.55 (m, 2H), 1.45 (br t, J=6.1 Hz, 2H), 1.32-1.16 (m, 3H), 0.89 (s, 6H). MS (ESI) m/z: 880 [M+H]⁺.

### Embodiment 5

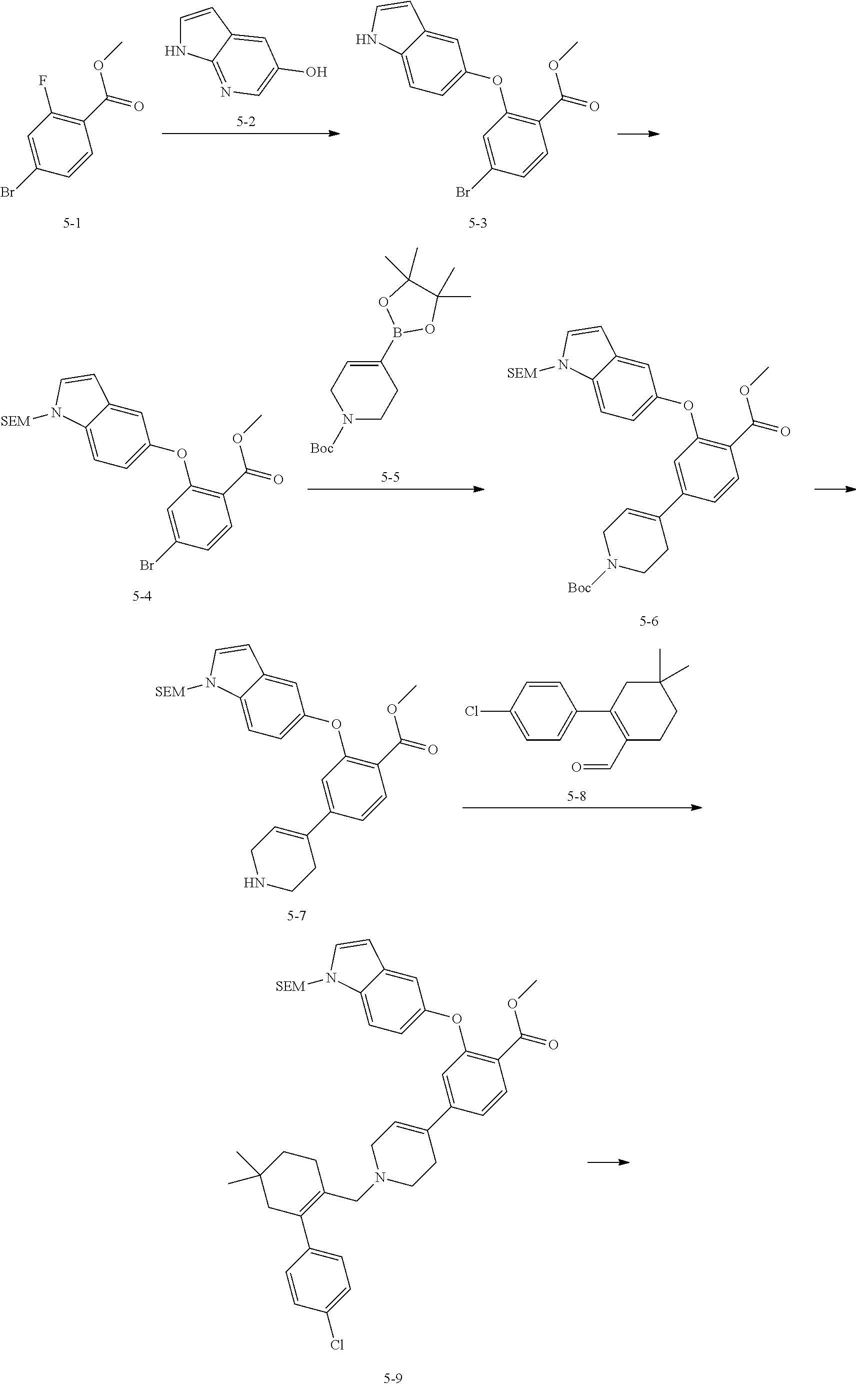

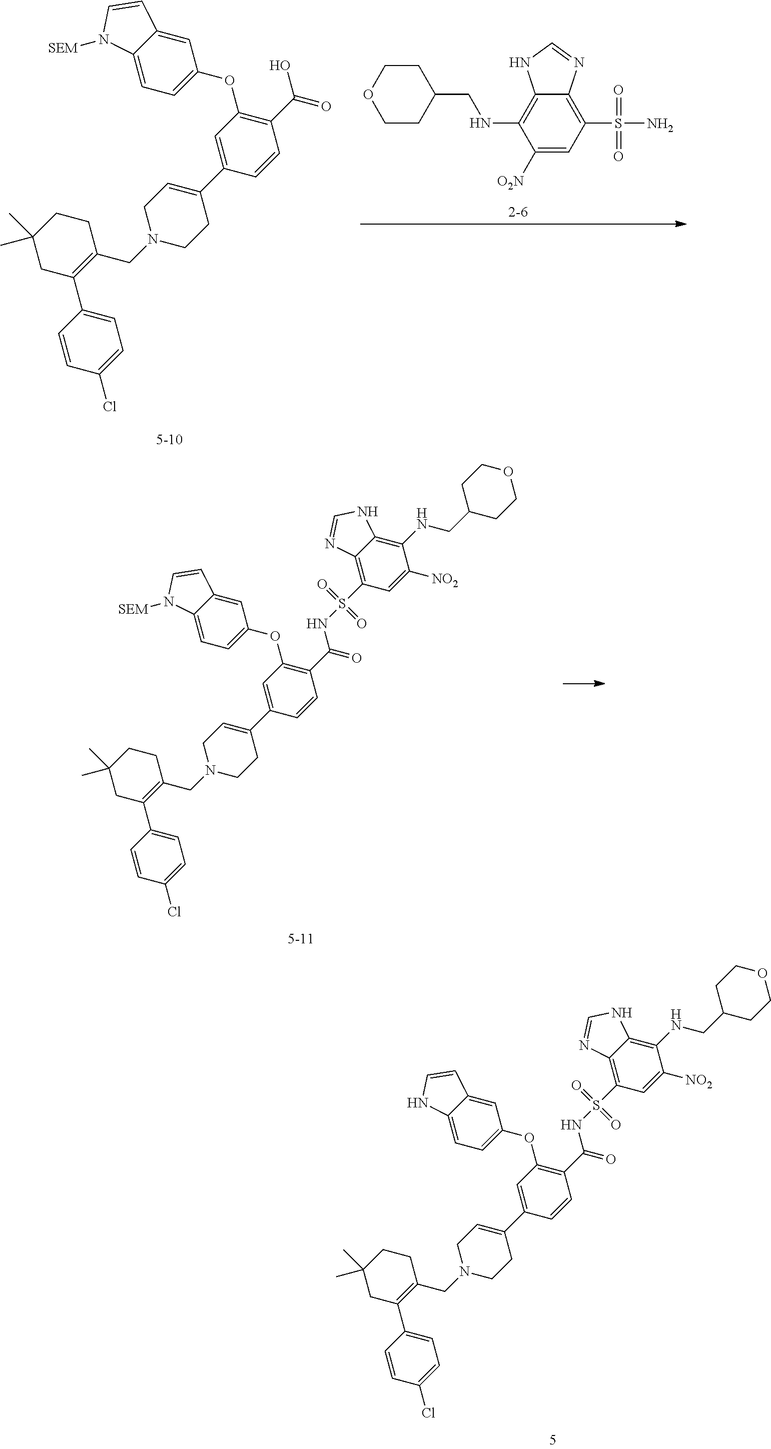 [SMILES: CC1(C)CCC(CN2CC=C(c3ccc(C(=O)O)c(Oc4ccc5c(ccn5COCC[Si](C)(C)C)c4)c3)CC2)=C(c2ccc(Cl)cc2)C1 ; NS(=O)(=O)c1cc([N+](=O)[O-])c(NCC2CCOCC2)c2[nH]cnc12 ; CC1(C)CCC(CN2CC=C(c3ccc(C(=O)NS(=O)(=O)c4cc([N+](=O)[O-])c(NCC5CCOCC5)c5[nH]cnc45)c(Oc4ccc5c(ccn5COCC[Si](C)(C)C)c4)c3)CC2)=C(c2ccc(Cl)cc2)C1 ; CC1(C)CCC(CN2CC=C(c3ccc(C(=O)NS(=O)(=O)c4cc([N+](=O)[O-])c(NCC5CCOCC5)c5[nH]cnc45)c(Oc4ccc5[nH]ccc5c4)c3)CC2)=C(c2ccc(Cl)cc2)C1]

### Step 1: Synthesis of Compound 5-3

Compound 5-1 (10 g, 42.91 mmol), compound 5-2 (5.76 g, 42.91 mmol) and potassium phosphate (18.22 g, 85.82 mmol) were addded to N,N-dimethylformamide (100 mL). The reaction solution was stirred at 100° C. for 3 hours. The reaction solution was quenched with water (500 mL) and extracted with ethyl acetate (200 mL) for three times. The organic phases were combined, dried, filtered and concentrated to obtain compound 5-3.

MS-ESI (m/z):346.9 [M+H]⁺.

### Step 2: Synthesis of Compound 5-4

Compound 5-3 (14 g, 40.33 mmol) was added to tetrahydrofuran (150 mL). 60% purity of sodium hydride (2.1 g, 52.42 mmol) was added at 0° C. and the mixture was stirred for 0.5 hours. 2-(Trimethylsilyl)ethoxymethyl chloride (10.76 g, 64.52 mmol) was added thereto. The reaction solution was stirred at 0° C. for 1 hour. The reaction solution was quenched with saturated aqueous ammonium chloride solution (200 mL) and extracted with ethyl acetate (150 mL) for three times. The combined organic phase was dried, filtered and concentrated to obtain compound 5-4.

¹H NMR (400 MHz, DMSO-d₆) δ=8.15 (d, J=2.5 Hz, 1H), 7.79 (d, J=8.5 Hz, 1H), 7.73 (dd, J=3.0, 4.5 Hz, 2H), 7.44 (dd, J=1.8, 8.5 Hz, 1H), 7.00 (d, J=1.8 Hz, 1H), 6.53 (d, J=3.5 Hz, 1H), 5.63 (s, 2H), 3.79 (s, 3H), 3.55-3.50 (m, 2H), 0.80 (d, J=7.8 Hz, 2H), 0.12 (s, 9H).

### Step 3: Synthesis of Compound 5-6

Compound 5-4 (12 g, 25.13 mmol), compound 5-5 (9.33 g, 30.13 mmol), 1,1′-bis(diphenylphosphino)ferrocene palladium dichloride (919.57 mg, 1.26 mmol) and potassium carbonate (6.95 g, 50.27 mmol) were added to 1,4-dioxane (200 mL) and water (50 mL). The reaction solution was heated to 100° C. and stirred for 16 hours under the protection of nitrogen. The reaction solution was diluted with dichloromethane (200 mL), washed with water (200 mL), and the organic phase was dried, filtered, concentrated and then subjected to silica gel column chromatography (petroleum ether/ethyl acetate=20/1 to 3/1) to obtain compound 5-6.

MS-ESI (m/z):580.5 [M+H]⁺.

### Step 4: Synthesis of Compound 5-7

Compound 5-6 (4 g, 6.90 mmol) and silica gel (41.45 g, 689.94 mmol) were added to toluene (150 mL). The reaction solution was stirred at 120° C. for 16 hours. The reaction solution was filtered, and the filtrate was concentrated, and subjected to silica gel column chromatography (dichloromethane/methanol=100/1 to 10/1) to obtain compound 5-7.

MS-ESI (m/z):480.5 [M+H]⁺.

### Step 5: Synthesis of Compound 5-9

Compound 5-7 (2 g, 4.17 mmol), compound 5-8 (1.14 g, 4.59 mmol) and zinc chloride (568.32 mg, 195.3 μmol) were addded to ethanol (40 mL). After the mixture was stirred at 25° C. for 30 minutes, sodium cyanoborohydride (786.11 mg, 12.51 mmol) was added thereto. The reaction was stirred at 50° C. for 2 hours. The reaction solution was quenched with water (100 mL) and extracted with dichloromethane (100 mL) for three times. The combined organic phase was dried, filtered, concentrated and then subjected to silica gel column chromatography (petroleum ether/ethyl acetate=15/1 to 1/1) to obtain compound 5-9.

¹H NMR (400 MHz, CDCl₃) δ=8.25 (d, J=2.5 Hz, 1H), 7.93 (d, J=8.3 Hz, 1H), 7.53 (d, J=2.5 Hz, 1H), 7.43 (d, J=3.5 Hz, 1H), 7.29 (s, 1H), 7.19 (dd, J=1.5, 8.0 Hz, 1H), 7.02 (d, J=8.5 Hz, 2H), 6.92 (d, J=1.5 Hz, 1H), 6.50 (d, J=3.5 Hz, 1H), 6.05 (br s, 1H), 5.72 (s, 2H), 3.90 (s, 3H), 3.66-3.58 (m, 2H), 2.98-2.89 (m, 4H), 2.45 (br d, J=4.8 Hz, 2H), 2.41 (br s, 2H), 2.27 (br s, 2H), 2.05 (s, 2H), 1.62 (s, 3H), 1.49 (t, J=6.4 Hz, 2H), 1.03-1.00 (m, 6H), 0.04 (s, 9H).

### Step 6: Synthesis of Compound 5-10

Compound 5-9 (1.4 g, 1.97 mmol) and lithium hydroxide monohydrate (247.4 mg, 5.9 mmol) were added to methanol (10 mL), tetrahydrofuran (10 mL) and water (5 mL). The reaction was stirred at 50° C. for 16 hours. The pH of the reaction solution was adjusted to 6 with 1 N hydrochloric acid, and the mixture was extracted with dichloromethane (30 mL) for three times. The combined organic phase was dried, filtered and concentrated to obtain compound 5-10.

MS-ESI (m/z):698.8 [M+H]⁺.

### Step 7: Synthesis of Compound 5-11

Compound 5-10 (900 mg, 1.29 mmol), compound 2-6 (503.77 mg, 1.42 mmol), 1-(3-dimethylaminopropyl)-3-ethylcarbodiimide hydrochloride (494.10 mg, 2.58 mmol), 4-dimethylaminopyridine (314.88 mg, 2.58 mmol) and triethylamine (260.81 mg, 2.58 mmol) were added to dichloromethane (20 mL). The reaction was stirred at 50° C. for 2 hours. The reaction solution was quenched with water (30 mL) and extracted with dichloromethane (20 mL) for three times. The organic phases were combined and concentrated to obtain a crude product, and the crude product was subjected to silica gel column chromatography (petroleum ether/ethyl acetate=12/1 to 1/2) to obtain compound 5-11.

MS-ESI (m/z):1035.6 [M+H]⁺.

### Step 8: Synthesis of Compound 5

Compound 5-11 (1 g, 0.965 mmol) and trifluoroacetic acid (5 mL) were added to dichloromethane (20 mL). The reaction solution was stirred at 15° C. for 16 hours. After the reaction solution was concentrated, ethanol (20 mL) and potassium carbonate (1.33 g, 9.62 mmol) were added thereto. The mixture was stirred at 20° C. for 2 hours. The reaction solution was quenched with water (50 mL) and extracted with dichloromethane (30 mL) for three times. The combined organic phase was concentrated and then subjected to silica gel column chromatography (dichloromethane/methanol=25/1 to 8/1) to obtain compound 5.

MS-ESI (m/z): 904.8 [M+H]⁺.

1H NMR (400 MHz, DMSO-d₆) δ=11.61 (br s, 1H), 9.34 (br t, J=6.4 Hz, 1H), 8.37 (br s, 1H), 8.07 (s, 1H), 7.94 (d, J=2.0 Hz, 1H), 7.56 (d, J=8.0 Hz, 1H), 7.45 (br d, J=12.3 Hz, 2H), 7.35 (br d, J=8.3 Hz, 2H), 7.15-7.02 (m, 3H), 6.71 (s, 1H), 6.34 (br s, 1H), 5.93 (br s, 1H), 4.20 (br t, J=6.5 Hz, 2H), 3.83 (br d, J=8.3 Hz, 2H), 3.32-3.18 (m, 8H), 2.35 (br d, J=17.6 Hz, 2H), 2.17 (br s, 2H), 2.04-1.97 (m, 3H), 1.93 (br s, 1H), 1.62 (br d, J=12.0 Hz, 2H), 1.41 (br s, 2H), 1.36-1.20 (m, 3H), 0.94 (s, 6H).

### Embodiment 6

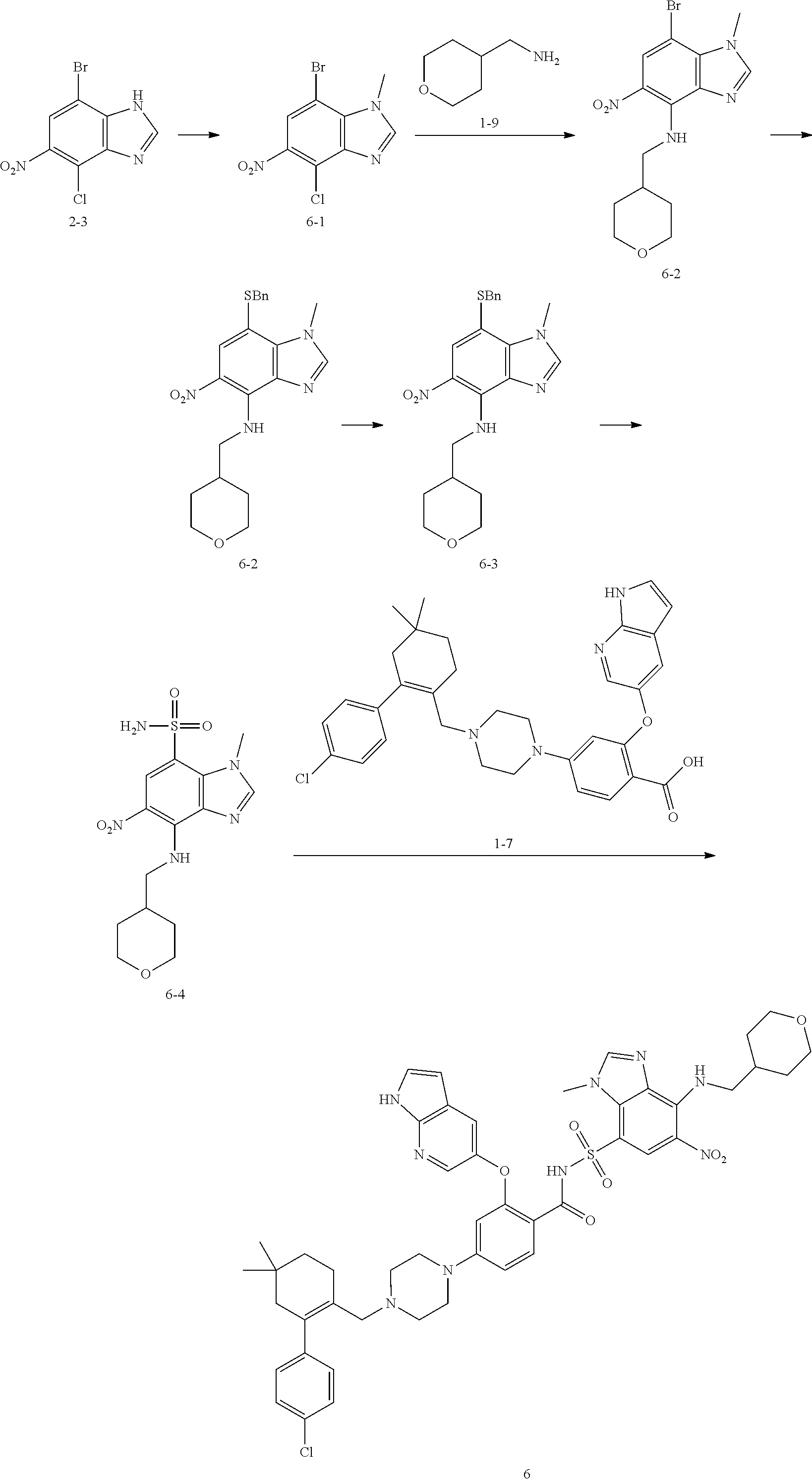

### Step 1: Synthesis of Compound 6-1

At 0° C., sodium hydride (2.24 g, 56.09 mmol, 60% purity) was addded to the N,N-dimethylformamide (50 mL) solution of compound 2-3 (15 g, 46.74 mmol) in batches. The mixture was stirred for 0.5 hours. Then methyl iodide (5.96 g, 41.97 mmol, 2.61 mL) was added dropwise. The mixture was stirred at 0° C. for 2 hours. The mixture was slowly added to water (150 mL). The obtained slurry was then filtered to obtain a crude product. The crude product was subjected to silica gel column chromatography (dichloromethane/methanol=40/1 to 10/1) to obtain compound 6-1.

¹H NMR (400 MHz, DMSO-d₆) δ=8.62 (s, 1H), 8.19 (s, 1H), 4.16 (s, 3H).

### Step 2: Synthesis of Compound 6-2

Compound 1-9 (8.11 g, 70.45 mmol) and diisopropylethylamine (9.11 g, 70.45 mmol, 12.27 mL) were added to the dimethyl sulfoxide (80 mL) solution of compound 6-1 (7.86 g, 23.47 mmol). The mixture was then heated to 90° C. and stirred for 10 hours. The mixture was poured into water (240 mL), stirred for 0.5 hours and filtered, and the obtain solid was dissolved in dichloromethane/methanol (10/1, 50 mL), washed with water (40 mL×2) and saturated brine (30 mL), respectively, and the organic phase was dried over anhydrous sodium sulfate and concentrated under reduced pressure. The crude product was purified by silica gel column chromatography (petroleum ether/ethyl acetate=2/1 to 1/1, 100 mL of dichloromethane was added per 300 mL of eluent) to obtain compound 6-2.

¹H NMR (400 MHz, DMSO-d₆) δ=9.20 (br t, J=6.2 Hz, 1H), 8.23 (s, 1H), 8.08 (s, 1H), 4.16 (t, J=6.5 Hz, 2H), 4.06 (s, 3H), 3.85 (br dd, J=3.1, 11.4 Hz, 2H), 3.28-3.22 (m, 2H), 1.92 (ddd, J=4.2, 7.2, 11.1 Hz, 1H), 1.62 (br d, J=12.2 Hz, 2H), 1.28 (dq, J=4.3, 12.2 Hz, 2H).

### Step 3: Synthesis of Compound 6-3

A mixture of compound 6-2 (900 mg, 2.44 mmol), benzyl mercaptan (605.52 mg, 4.88 mmol, 571.24 μL), Pd₂(dba)₃ (223.22 mg, 243.76 mmol), Xantphos (282.09 mg, 487.5 mmol) and diisopropylethylamine (945.13 mg, 7.31 mmol, 1.27 mL) in toluene (10 mL) was replaced with nitrogen for three times, then the mixture was stirred at 110° C. for 10 hours under nitrogen atmosphere. The mixture was diluted with toluene (30 mL) and filtered. The filtrate was washed with saturated ammonium chloride solution (20 mL) and saturated brine (10 mL), dried over anhydrous sodium sulfate and concentrated under reduced pressure. The crude product was purified by silica gel column chromatography (petroleum ether/ethyl acetate=3/1 to 2/1) to obtain compound 6-3.

¹H NMR (400 MHz, DMSO-d₆) δ=9.20 (br t, J=6.0 Hz, 1H), 8.13 (s, 1H), 7.87 (s, 1H), 7.26-7.19 (m, 3H), 7.14 (br d, J=6.8 Hz, 2H), 4.18 (br t, J=6.3 Hz, 2H), 4.07 (s, 2H), 4.00 (s, 3H), 3.86 (br d, J=8.9 Hz, 2H), 3.30-3.22 (m, 2H), 1.92 (br d, J=4.4 Hz, 1H), 1.62 (br d, J=12.8 Hz, 2H), 1.36-1.25 (m, 2H).

### Step 4: Synthesis of Compound 6-4

At 0° C., N-chlorosuccinimide (1.94 g, 14.55 mmol) was added to the acetic acid (20 mL) solution of compound 6-3 (2 g, 4.85 mmol) in batches. The mixture was stirred at 0° C. for 2 hours, then heated to 28° C., and stirred for another 10 hours. Then N-chlorosuccinimide (129.49 mg, 969.69 mmol) was added in batches at 0° C., and the mixture was stirred for 1 hour. The mixture was then heated to 28° C. and stirred for another 1 hour. The mixture was then added dropwise to ammonia water (40 mL) at 0° C. and stirred for 1 hour, then the mixture was stirred at 28° C. for 1 hour. The mixture was diluted with dichloromethane (40 mL). The organic phase was then separated and the aqueous phase was extracted with dichloromethane/methanol (10/1, 20 mL×3). The combined organic phase was washed with saturated ammonium chloride solution (10 mL×2) and brine (10 mL), dried over anhydrous sodium sulfate and concentrated under reduced pressure. The residue was purified by silica gel column chromatography (dichloromethane/methanol=120/1 to 80/1) to obtain compound 6-4.

¹H NMR (400 MHz, DMSO-d₆) δ=9.39 (br t, J=6.2 Hz, 1H), 8.59 (s, 1H), 8.28 (s, 1H), 7.51-7.17 (m, 2H), 4.23 (br t, J=6.5 Hz, 2H), 4.12 (s, 3H), 3.85 (br dd, J=3.1, 11.1 Hz, 2H), 3.28-3.21 (m, 2H), 1.98-1.88 (m, 1H), 1.64 (br d, J=12.6 Hz, 2H), 1.37-1.24 (m, 2H).

### Step 5: Synthesis of Compound 6

Triethylamine (523.59 mg, 5.17 mmol, 720.21 μL) and compound 1-7 (1.24 g, 2.17 mmol) were added to the dichloromethane (13.0 mL) solution of compound 6-4 (1.26 g, 2.07 mmol), 1-(3-dimethylaminopropyl)-3-ethylcarbodiimide hydrochloride (595.16 mg, 3.10 mmol) and 4-dimethylaminopyridine (632.15 mg, 5.17 mmol). Then the mixture was stirred at 45° C. for 10 hours. The mixture was diluted with dichloromethane (30 mL), washed with saturated aqueous ammonium chloride solution (10 mL×2) and saturated brine (10 mL), dried over anhydrous sodium sulfate and concentrated under reduced pressure. The crude product was purified by prep_HPLC (neutral system) (chromatographic column: Kromasil Eternity XT 250*80 mm*10 μm; mobile phase: [water (10 mmol ammonium bicarbonate)-acetonitrile]; B (acetonitrile) %: 35% to 65%, 25 min) to obtain compound 6.

¹H NMR (400 MHz, DMSO-d₆) δ=11.55 (br s, 1H), 9.30 (br t, J=6.3 Hz, 1H), 8.54 (br s, 1H), 8.04 (s, 1H), 7.89 (br s, 1H), 7.57 (d, J=8.8 Hz, 1H), 7.44 (br s, 1H), 7.35 (d, J=8.4 Hz, 3H), 7.05 (d, J=8.3 Hz, 2H), 6.62 (dd, J=2.1, 8.8 Hz, 1H), 6.33 (br s, 1H), 6.16 (s, 1H), 4.20-4.16 (m, 3H), 4.16 (br s, 3H), 3.83 (br dd, J=3.0, 11.3 Hz, 2H), 3.28-3.20 (m, 2H), 2.99 (br s, 4H), 2.76 (br d, J=3.4 Hz, 2H), 2.29-2.10 (m, 6H), 1.96 (br s, 2H), 1.91 (td, J=3.6, 7.6 Hz, 1H), 1.62 (br d, J=11.1 Hz, 2H), 1.39 (br t, J=6.3 Hz, 2H), 1.33-1.22 (m, 2H), 0.93 (s, 6H). MS (ESI) m/z: 922 [M+H]⁺.

### Embodiment 7

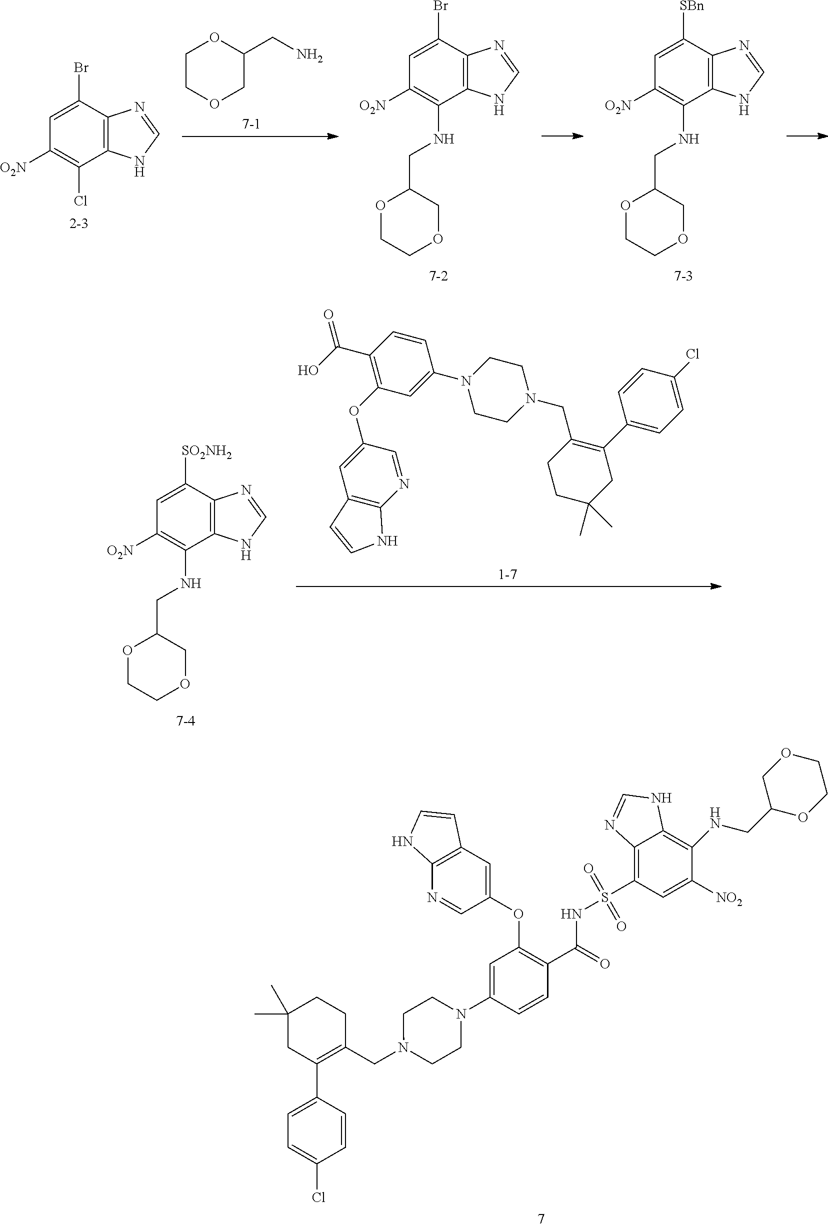

### Step 1: Synthesis of Compound 7-2

Potassium carbonate (1.94 g, 14.02 mmol) and compound 7-1 (1.64 g, 14.02 mmol) were added to the dimethyl sulfoxide (20 mL) solution of compound 2-3 (1.5 g, 4.67 mmol) under the protection of nitrogen. The reaction solution was stirred at 120° C. for 16 hours. The reaction solution was added with water (100 mL), and a large amount of solid was precipitated, and then the mixture was filtered, and the filter cake was dried to obtain compound 7-2.

¹H NMR (400 MHz, DMSO-d₆): δ=13.39 (br s, 1H), 9.23 (br t, J=5.8 Hz, 1H), 8.29 (s, 1H), 8.10 (s, 1H), 4.46-4.55 (m, 1H), 4.07-4.16 (m, 1H), 3.83-3.88 (m, 1H), 3.80 (br d, J=11.8 Hz, 2H), 3.57-3.68 (m, 2H), 3.45-3.52 (m, 1H), 3.34-3.39 (m, 1H); LCMS (ESI) m/z: 357/359 [M+H]⁺.

### Step 2: Synthesis of Compound 7-3

A mixture of compound 7-2 (600.00 mg, 1.68 mmol), benzyl mercaptan (313 mg, 2.52 mmol), Pd₂(dba)₃ (61.4 mg, 84 μmol), Xantphos (50.2 mg, 84 μmol) and diisopropylethylamine (651.37 mg, 5.04 mg) in toluene (12 mL) was replaced with nitrogen for three times, then stirred at 110° C. for 16 hours under nitrogen atmosphere. The reaction mixture was poured into water (50 mL), extracted with ethyl acetate (30 mL×3). The organic phase was washed with saturated brine (30 mL), dried over anhydrous sodium sulfate, filtered and the filtrate was concentrated under reduced pressure, and the residue was purified by silica gel column chromatography (petroleum ether/ethyl acetate=1/0 to 5/1) to obtain compound 7-3.

¹H NMR (400 MHz, DMSO-d₆): δ=13.20 (br s, 1H), 9.25 (br t, J=5.3 Hz, 1H), 8.24 (s, 1H), 7.81 (s, 1H), 7.15-7.26 (m, 5H), 4.52 (br d, J=11.8 Hz, 1H), 4.13 (br d, J=7.0 Hz, 1H), 4.10 (s, 2H), 3.83-3.87 (m, 1H), 3.80 (br d, J=11.3 Hz, 2H), 3.56-3.68 (m, 2H), 3.44-3.53 (m, 1H), 3.33-3.37 (m, 1H); MS-ESI (m/z):401 [M+H]⁺.

### Step 3: Synthesis of Compound 7-4

At 0° C., N-chlorosuccinimide (448.66 mg, 3.36 mmol) was added to a mixture of compound 7-3 (450 mg, 1.12 mmol), acetonitrile (9 mL), acetic acid (0.9 mL) and water (1.8 mL) in batches. The mixture was stirred at 25° C. for 32 hours. Then, the reaction mixture was added to ammonia water (16.38 g, 116.82 mmol, 6.00 mL, 25% purity) and stirred at 25° C. for 2 hours. The reaction mixture was diluted with water (100 mL), extracted with ethyl acetate (100 mL×3). The combined organic phase was washed with saturated brine (50 mL), dried over anhydrous sodium sulfate, filtered, and the filtrate was concentrated under reduced pressure to obtain a residue, and the residue was purified by thin layer chromatography (silica gel, petroleum ether/ethyl acetate=1/2) to obtain compound 7-4.

¹H NMR (400 MHz, DMSO-d₆): δ=12.84 (br s, 1H), 9.39 (br t, J=5.8 Hz, 1H), 8.43 (s, 1H), 8.29 (s, 1H), 7.53 (br s, 2H), 4.52-4.61 (m, 1H), 4.14-4.23 (m, 1H), 3.85-3.90 (m, 1H), 3.78-3.85 (m, 2H), 3.57-3.69 (m, 2H), 3.45-3.53 (m, 1H), 3.35-3.40 ppm (m, 1H); MS(ESI) m/z: 358 [M+H]⁺.

### Step 4: Synthesis of Compound 7

Compound 7-4 (0.3 g, 0.840 mmol), compound 1-7 (0.480 g, 0.840 mmol), 4-dimethylaminopyridine (0.206 g, 1.68 mmol), 1-(3-dimethylaminopropyl)-3-ethylcarbodiimide hydrochloride (0.322 g, 1.68 mmol) and triethylamine (0.170 g, 1.68 mmol) were added to dichloromethane (20 mL), and the mixture was stirred at 45° C. for 16 hours. The reaction solution was poured into 20 mL of water, extracted with dichloromethane (20 mL×3), washed with saturated brine (20 mL×2), and finally dried over anhydrous sodium sulfate. After dichloromethane was evaporated to dryness by rotary evaporation, the crude product was purified by silica gel column chromatography (dichloromethane/methanol=50/1 to 8/1) to obtain compound 7.

¹H NMR (400 MHz, DMSO-d₆): δ=12.90 (br s, 1H), 11.70 (br s, 1H), 9.36-9.43 (m, 1H), 8.52 (br s, 1H), 8.22 (br s, 1H), 8.05 (br s, 1H), 7.49-7.63 (m, 3H), 7.34 (d, J=8.0 Hz, 2H), 7.03 (d, J=8.3 Hz, 2H), 6.65 (br d, J=8.8 Hz, 1H), 6.40 (br s, 1H), 6.15 (s, 1H), 5.76 (s, 1H), 4.54 (br dd, J=8.0, 3.8 Hz, 1H), 4.11-4.23 (m, 1H), 3.75-3.95 (m, 4H), 3.64 (br t, J=11.0 Hz, 2H), 3.43-3.59 (m, 2H), 3.35-3.41 (m, 2H), 3.07 (br s, 4H), 2.78 (br s, 1H), 2.23 (br s, 2H), 2.14 (br s, 2H), 1.92-1.99 (m, 2H), 1.33-1.43 (m, 2H), 0.92 ppm (s, 6H); MS(ESI) m/z: 910 [M+H]⁺.

### Embodiment 8

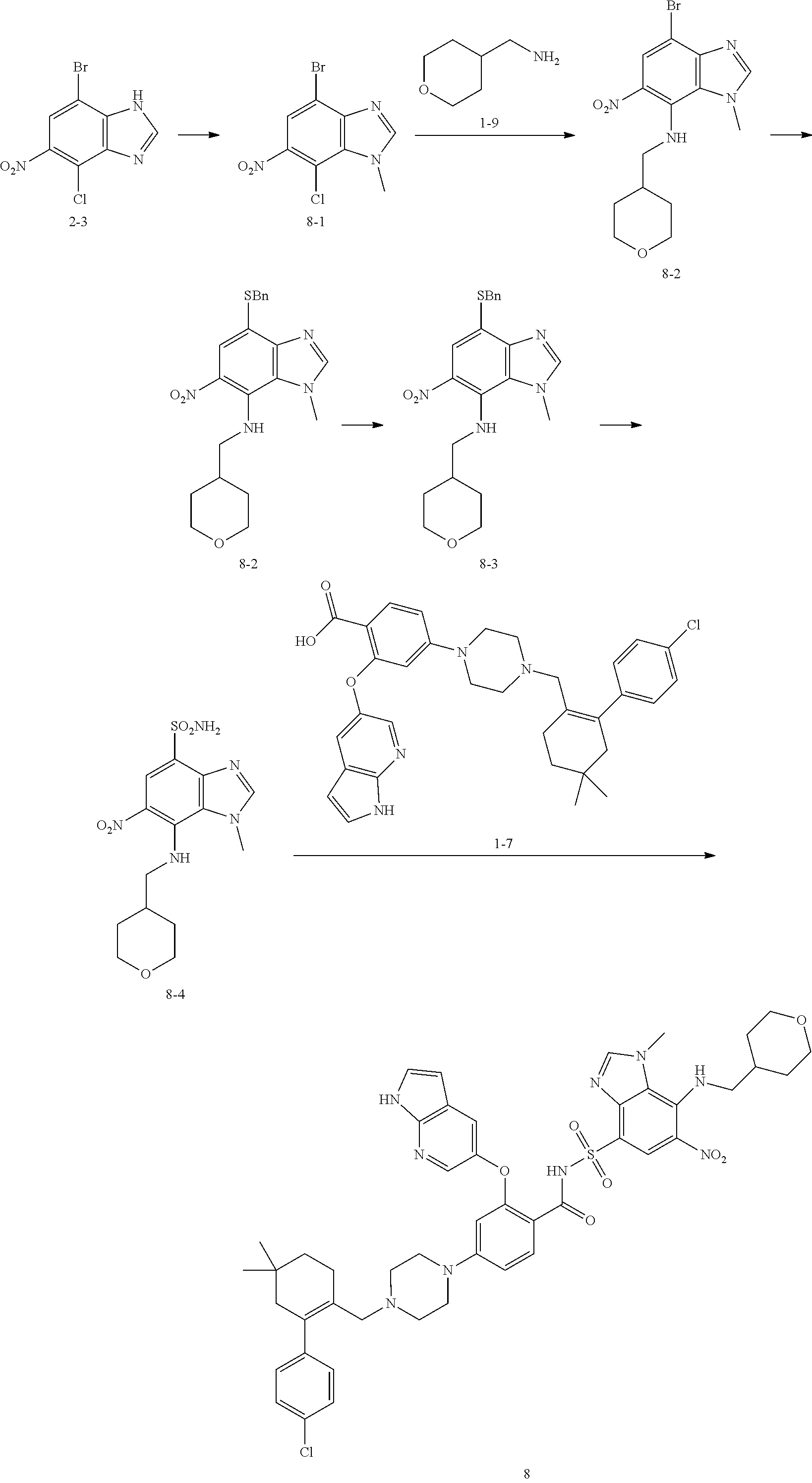

### Step 1: Synthesis of Compound 8-1

Potassium carbonate (2.84 g, 20.6 mmol) and iodomethane (1.36 g, 9.6 mmol) were added to the tetrahydrofuran (22 mL) solution of compound 2-3 (2.2 g, 6.86 mmol). The reaction solution was stirred at 15° C. for 16 hours. The reaction solution was addded to saturated ammonium chloride solution (50 mL) and extracted with ethyl acetate (30 mL×3). The organic phases were combined, dried, filtered and concentrated to obtain compound 8-1. LCMS (ESI) m/z: 334/336[M+H]⁺.

### Step 2: Synthesis of Compound 8-2

Diisopropylethylamine (2.27 g, 17.56 mmol, 3.06 mmol) and tetrahydropyran-4-yl-methylamine (2.04 g, 17.69 mmol) were added to the dimethyl sulfoxide (20 mL) solution of compound 8-1 (790.00 mg, 2.36 mmol). The mixture was stirred at 90° C. for 11 hours. The reaction mixture was poured into 100 mL of water, filtered, and the filter cake was washed with 30 mL of water, and concentrated under reduced pressure and dried to obtain a residue. The residue was purified by silica gel column chromatography (petroleum ether/ethyl acetate=10/1 to 0/1) to obtain compound 8-2.

¹H NMR (400 MHz, DMSO-d₆) δ=8.46 (s, 1H), 8.02 (s, 1H), 6.67 (br t, J=6.2 Hz, 1H), 4.09 (s, 3H), 3.81 (br dd, J=3.2, 11.2 Hz, 2H), 3.26-3.14 (m, 4H), 1.79-1.65 (m, 1H), 1.50 (br d, J=12.5 Hz, 2H), 1.20-1.08 (m, 2H).

### Step 3: Synthesis of Compound 8-3

A mixture of compound 8-2 (840.00 mg, 2.28 mmol), benzyl mercaptan (565.15 mg, 4.55 mmol), Pd₂(dba)₃ (208.34 mg, 227.51 μmol), Xantphos (263.28 mg, 455.02 μmol) and diisopropylethylamine (882.13 mg, 6.83 mmol, 1.19 mL) in toluene (9 mL) was replaced with nitrogen for three times, then the mixture was stirred at 110° C. for 16 hours under nitrogen atmosphere. The reaction mixture was diluted with 20 mL of water and extracted with ethyl acetate (40 mL×3). The combined organic phase was washed with saturated brine (10 mL×3), dried over anhydrous sodium sulfate, filtered, and the filtrate was concentrated under reduced pressure to obtain a residue. The residue was purified by silica gel column chromatography (petroleum ether/ethyl acetate=10/1 to 1/4) to obtain compound 8-3.

¹H NMR (400 MHz, DMSO-d₆) δ=8.41 (s, 1H), 7.65 (s, 1H), 7.34-7.18 (m, 5H), 6.62 (t, J=6.3 Hz, 1H), 4.38 (s, 2H), 4.08 (s, 3H), 3.80 (br dd, J=3.2, 11.2 Hz, 2H), 3.26-3.17 (m, 2H), 3.14 (t, J=6.4 Hz, 2H), 1.77-1.62 (m, 1H), 1.49 (br d, J=12.7 Hz, 2H), 1.13 (dq, J=4.3, 12.3 Hz, 2H).

### Step 4: Synthesis of Compound 8-4

At 0° C., N-chlorosuccinicimide (113.30 mg, 848.48 μmol) was added to the acetic acid (1 mL) and water (0.25 mL) solution of compound 8-3 (100 mg, 242.42 μmol). The temperature of the mixture was raised to 15° C. and the mixture was stirred for 16 hours. The mixture was added to ammonia water (2.73 g, 21.81 mmol, 3 mL, 25% purity) at 0° C. The mixture was stirred at 15° C. for 1 hour. The reaction mixture was diluted with 5 mL of water and extracted with ethyl acetate (20 mL×3). The combined organic phase was washed with saturated brine (5 mL×3), dried over sodium sulfate, filtered, and the filtrate was concentrated under reduced pressure to obtain a residue. The residue was purified by preparative chromatography (silica gel, ethyl acetate/ethanol=10/1) to obtain compound 8-4.

MS (ESI) m/z: 370 [M+H]⁺.

### Step 5: Synthesis of Compound 8

Compound 1-7 (51.02 mg, 84.34 μmol) and triethylamine (24.87 μL, 162.24 μmol) were added to the dichloromethane (2 mL) solution of compound 8-4 (33 mg, 89.34 μmol), 1-(3-dimethylaminopropyl)-3-ethylcarbodiimide hydrochloride (20.55 mg, 107.20 μmol) and 4-dimethylaminopyridine (21.83 mg, 178.67 μmol). The mixture was stirred at 40° C. for 16 hours. The mixture was concentrated under reduced pressure, and the residue was purified by high performance liquid chromatography (column: Welch Xtimate C18 100*40 mm*3 μm; mobile phase: [water (0.225% trifluoroacetic acid)-acetonitrile]; B (acetonitrile)%: 38%-68%, 8.5 minutes) to obtain compound 8 (trifluoroacetate).

¹H NMR (400 MHz, DMSO-d₆) δ=11.83-11.55 (m, 1H), 8.33 (br s, 2H), 8.08-7.92 (m, 1H), 7.67-7.42 (m, 3H), 7.38-7.27 (m, 3H), 7.03 (d, J=8.4 Hz, 3H), 6.63 (dd, J=1.9, 8.7 Hz, 1H), 6.41 (br s, 1H), 6.13 (s, 1H), 4.04 (s, 3H), 3.79 (br dd, J=3.0, 11.3 Hz, 2H), 3.25-3.18 (m, 4H), 3.02 (br s, 4H), 2.70 (s, 2H), 2.14 (br d, J=3.4 Hz, 6H), 1.94 (br s, 2H), 1.84-1.70 (m, 1H), 1.54-1.43 (m, 2H), 1.37 (br t, J=6.4 Hz, 2H), 1.20-1.08 (m, 2H), 0.92 (s, 6H). MS (ESI) m/z: 922 [M+H]⁺.

### Embodiment 9

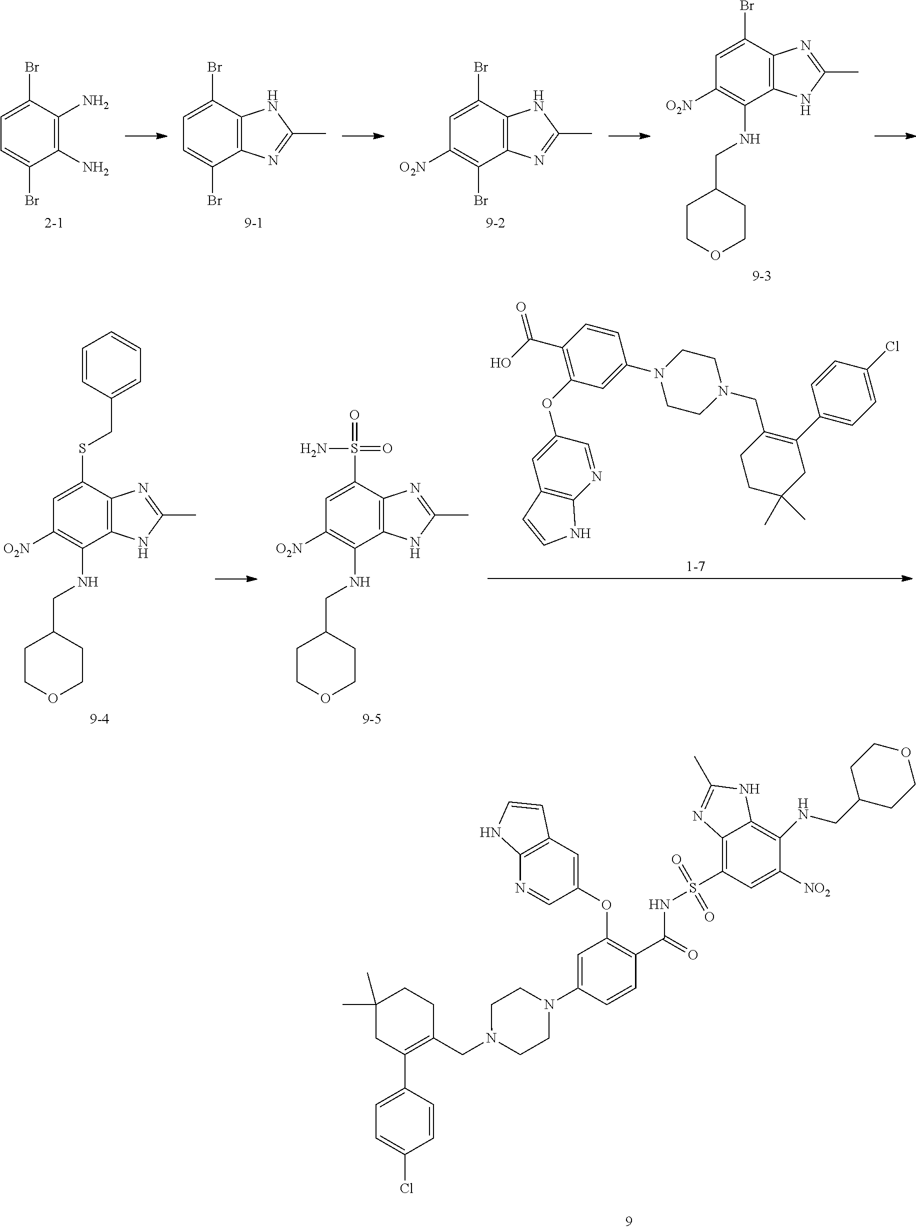

### Step 1: Synthesis of Compound 9-1

1,1,1-Trimethoxyethane (1.36 g, 11.28 mmol) was added to the tetrahydrofuran (40 mL) solution of compound 2-1 (2 g, 7.52 mmol) and p-toluenesulfonic acid (129.51 mg, 0.752 mmol). The reaction solution was stirred at 20° C. for 1 hour. The reaction solution was added with water (50 mL) and extracted with ethyl acetate (30 mL) for three times. The organic phases were combined, dried, filtered and concentrated to obtain compound 9-1.

LCMS (ESI) m/z: 289/291[M+H]⁺.

### Step 2: Synthesis of Compound 9-2

Potassium nitrate (345.2 mg, 3.41 mmol) was added to the sulfuric acid (10 mL, 98%) solution of compound 9-1(0.9 g, 3.1 mmol) at 0° C. The mixture was stirred at 0° C. for 1 hour. The mixture was poured into the mixture of ice water (50 mL) and ammonia water (25 mL), filtered, and the filter cake was collected and dried under vacuum to obtain compound 9-2.

LCMS (ESI) m/z: 334/336[M+H]⁺.

### Step 3: Synthesis of Compound 9-3

Potassium carbonate (1.24 g, 8.97 mmol) and tetrahydropyran-4-yl-methylamine (1.03 g, 8.97 mmol) were added to the dimethyl sulfoxide (10 mL) solution of compound 9-2 (1.0 g, 2.99 mmol). The mixture was stirred at 100° C. for 16 hours. The reaction mixture was poured into 100 mL of water and extracted with ethyl acetate (50 mL×3). The organic phases were combined, dried over anhydrous sodium sulfate and concentrated to obtain compound 9-3.

LCMS (ESI) m/z: 369/371[M+H]⁺.

### Step 4: Synthesis of Compound 9-4

A mixture of compound 9-3 (0.5 g, 1.35 mmol), benzyl mercaptan (238.02 μL, 2.03 mmol), Pd₂(dba)₃ (124 mg, 135 μmol), Xantphos (117.54 mg, 203 μmol) and diisopropylethylamine (471.11 μL, 2.71 mmol) in toluene (10 mL) was replaced with nitrogen for three times, then stirred at 110° C. for 16 hours under nitrogen atmosphere. The reaction mixture was diluted with 50 mL of water and extracted with ethyl acetate (40 mL×3). The combined organic phases were washed with saturated brine (30 mL), dried over anhydrous sodium sulfate, filtered, and the filtrate was concentrated under reduced pressure to obtain a residue. The residue was purified by silica gel column chromatography (petroleum ether/ethyl acetate=10/1 to 1/4) to obtain compound 9-4.

MS (ESI) m/z: 413 [M+H]⁺.

### Step 5: Synthesis of Compound 9-5

At 0° C., N-chlorosuccinicimide (647.43 mg, 4.85 mmol) was added in batches to the acetic acid (4 mL) and water (1 mL) solution of compound 9-4 (500 mg, 1.21 mmol) in batches at 0° C. The mixture was stirred at 25° C. for 32 hours. The reaction mixture was then added to ammonia water (20 mL, 116.82 mmol, 25% purity) and the mixture was stirred at 25° C. for 2 hours. The reaction mixture was diluted with water (100 mL), extracted with ethyl acetate (100 mL×3). The combined organic phase was washed with saturated brine (50 mL), dried over anhydrous sodium sulfate, filtered, and the filtrate was concentrated under reduced pressure to obtain a residue, and the residue was purified by thin layer chromatography (silica gel, petroleum ether/ethyl acetate=1/2) to obtain compound 9-5.

MS (ESI) m/z: 370 [M+H]⁺.

### Step 6: Synthesis of Compound 9

Triethylamine (75.36 μL, 0.54 mmol) and compound 1-7 (0.15 g, 0.27 mmol) were added to the dichloromethane (10 mL) solution of compound 9-5 (0.1 g, 0.27 mmol), 1-(3-dimethylaminopropyl)-3-ethylcarbodiimide hydrochloride (103.8 mg, 0.54 mmol) and 4-dimethylaminopyridine (66.1 mg, 0.54 mmol). Then the mixture was stirred at 45° C. for 10 hours. The mixture was diluted with dichloromethane (30 mL), washed with saturated aqueous ammonium chloride solution (10 mL×2) and saturated brine (10 mL), dried over anhydrous sodium sulfate and concentrated under reduced pressure. The crude product was purified by high performance liquid chromatography (column: Welch Xtimate C18 100*40 mm*3 μm; mobile phase: [water (0.075% trifluoroacetic acid)-acetonitrile]; B (acetonitrile)%: 45%−75%, 7 minutes) to obtain compound 9 (trifluoroacetate).

¹H NMR (400 MHz, DMSO-d₆) δ=12.72 (br s, 1H), 11.80 (br s, 1H), 11.38 (br s, 1H), 9.30 (br s, 2H), 8.52 (br s, 1H), 8.09 (br s, 1H), 7.66 (br s, 1H), 7.57 (br s, 2H), 7.39 (br s, 2H), 7.09 (br s, 2H), 6.72 (br s, 1H), 6.45 (br s, 1H), 6.19 (br s, 1H), 3.85 (br s, 4H), 3.73-3.48 (m, 5H), 3.26 (br s, 3H), 3.02 (br s, 2H), 2.74 (br s, 1H), 2.45-2.28 (m, 3H), 2.19 (br s, 2H), 2.12-1.89 (m, 3H), 1.63 (br s, 2H), 1.46 (br s, 2H), 1.31 (br s, 2H), 0.95 (br s, 6H); MS-ESI (m/z): 922.3 [M+H]⁺.

Biological Test Data:

### Experimental Embodiment 1: In Vitro Test of Inhibitory Effect of the Compound on Bcl-2/Bcl-xL Protease (Enzymology Experiment)

This experiment was based on the competition between fluorescently-labeled Bak/Bad/Noxa peptides and GST-labeled Bcl family proteins. The fluorescence detection method based on HTRF was to observe the binding degree by using the fluorescence ratio between Tb-labeled anti-GST and FAM-labeled peptides. This peptide binds to the surface of the Bcl family protein pocket, which is essential for its anti-apoptotic function.

1.1 Experimental reagents: analysis buffer: 20 mM potassium phosphate, pH 7.5, 50 mM sodium chloride, 1 mM EDTA, 0.005% Tritonx-100 and 1% DMSO.

1.2 Probe: 5,6-FAM-peptide

1.3 Target:

Bcl-2: APT-11-441 of RBC category

Human recombinant Bcl-2 (amino acid 1-207) (GenBank accession number: NM_000663), with C-term GST tag, MW=49.2 kDa, expressed in E. coli system.

Bcl-xL: APT-11-442 of RBC category

Human recombinant Bcl-xL (amino acid 1-209) (GenBank accession number: Z23115), with C-term GST tag, MW=49.78 kDa, expressed in E. coli system.

Human recombinant Bcl-2 (amino acid 171-327) (GenBank accession number: NM_021960), with C-term GST tag, MW=44.4 kDa, expressed in E. coli system.

1.4 Experimental Conditions:

4 nM Bcl-2 and 100 nM FAM-BAK

3 nM Bcl-xL and 40 nM FAM-Bad

1.5 Reference Compound

ABT-737 (or ABT-263) and ABT-199

1.6 Experimental Steps

a) The Bcl enzyme reaction solution was prepared in the newly prepared analysis buffer;

b) the Bcl enzyme reaction solution was provided;

c) the compound was first prepared into a 100% dimethyl sulfoxide solution, and then the compound solution was mixed with the Bcl enzyme reaction solution using (Echo550; nanoliter range) technology, and co-incubated for 10 minutes;

d) the FAM peptide solution was added.

e) the mixed solution was gently stirred in dark and room temperature, and co-cultured for 10 minutes.

f) Anti-GST solution was added.

g) The mixed solution was gently stirred in dark and room temperature, and co-cultured for 1 hour.

h) The HTRF high-frequency fluorescence ratio was tested, and the IC₅₀ values were calculated.

The experimental results are shown in table 1:

TABLE 1
IC₅₀ test results of HTRF detection

| | Enzyme IC₅₀ (nM) | |
|---|---|---|
| Compound | IC₅₀ (nM) for inhibition | IC₅₀ (nM) for inhibition |
| number | of Bcl-2 enzyme | of Bcl-xL enzyme (nM) |
| ABT-199 | 2.2 | 83 |
| Compound 2 | 1.5 | 411 |
| Compound 5 | 2.6 | 504 |
| Compound 6 | 2.4 | 173 |
| Compound 8 | 1.3 | 1110 |
| Compound 9 | 3.6 | 326 |

Conclusion: the results show that compared with anti-apoptotic Bcl-2 protein and anti-apoptotic Bcl-xL protein, the compound of the present disclosure has a significant inhibitory effect on the anti-apoptotic Bcl-2 protein, and the inhibitory effect on the anti-apoptotic Bcl-xL protein is significantly weaker than that of ABT-199, and the target selectivity is higher.

### Experimental Embodiment 2: In Vitro Test of Inhibitory Effect of the Compound on RS4; 11 Cell Proliferation (Cell Experiment)

2.1 Experimental objectives: To obtain the IC₅₀ value of the test compound in RS4;11 cell lines;

2.2 incubation time: 72 hours;

2.3 experimental method: CTG (Cell Titer-Glo™ Luminescent Cell Viability Assay);

2.4 experimental steps:

a. when the cells were fused to 80%, the cells were collected and counted;

b. RS4; 11 cell suspension was diluted to 5000 cells/well and 20 uls of cell suspension was seeded in each well of 384-well plate;

c. the cell plates were placed back to 37° C. and incubated in a 5% carbon dioxide incubator for 24 hours;

d. the test compound was prepared into DMSO solution, and 5 μL of each was added to the designated well of the test plate, and the final concentration of DMSO solution was 0.5%;

e. initiation cell viability was detected by CTG;

f. the test plate was put back into the incubator and incubated for another 72 hours;

g. after 72 hours incubation, CellTiter-Glo™ luminescent cell viability assay was completed according to the manufacturer's manual;

h. data calculation:

$$Inhibition ratio \% = \frac{compound RFU - average ( negative control RFU )}{average ( initial RFU ) - average ( negative control RFU )} \times 100 \%$$

The experimental results are shown in table 2:

TABLE 2
IC₅₀ test results of CTG detection for inhibition
of RS4; 11 cell proliferation

| Compound | IC₅₀ (nM) for inhibition of |
|---|---|
| number | RS4; 11 cell proliferation |
| Compound 1 | 1.4 |
| Compound 2 | 2.7 |
| Compound 5 | 10.2 |
| Compound 6 | 4.6 |
| Compound 7 | 6.8 |
| Compound 8 | 6.5 |
| Compound 9 | 1.9 |
| / | / |

Conclusion: The results show that the compounds of the present disclosure have a significant inhibitory effect on the division and proliferation of RS4;11 cells.

### Experimental Embodiment 3: Metabolic Stability Evaluation of Liver Microsomal In Vitro

3.1 Preparation of test samples and control working solution: 5 μL of the dimethyl sulfoxide solution (10 mM) of compound 2 was diluted with 495 μL of acetonitrile, and the resulting working solution concentration was: 100 μM, 99% acetonitrile;

3.2 preparation of nicotinamide adenine dinucleotide phosphate cofactor solution: an appropriate amount of NADPH powder was weighed and diluted into 10 mM magnesium chloride solution (working solution concentration: 10 unit/mL; final concentration of reaction system: 1 unit/mL);

3.3 preparation of liver microsomes: an appropriate concentration working solution of liver microsomes (human, SD rat, CD-1 mouse, beagle dog) was prepared in 100 mM potassium phosphate buffer;

3.4 preparation of quenching solution: cold (4° C.) acetonitrile containing 200 ng/mL tolbutamide and 200 ng/mL labetalol as internal standard (IS) was used as quenching solution;

3.5 experimental operation:

a. empty “incubation” plates T60 and NCF60 were preheated for 10 minutes;

b. the liver microsomes were diluted to 0.56 mg/mL with 100 mM phosphate buffer;

c. 445 μL of microsomal working solution (0.56 mg/mL) was transferred to the preheated “incubation” plates T60 and NCF60, and then the“incubation” plates T60 and NCF60 were preincubated for 10 min with continuous shaking at 37° C. 54 μL of liver microsomes were transferred to a blank plate, and 6 μL of NAPDH cofactor was added to the blank plate, and then 180 μL of quenching solution was added thereto;

d. 5 μL of the dimethyl sulfoxide (100 μM) solution of compound 2 was added to the “incubation” plate (T60 and NCF60) containing microsomes and the mixture was mixed fully for three times;

e. for NCF60 plate, 50 μL of buffer was added thereto and the mixture was fully mixed for three times. Timing was started; the plate was shaken at 37° C. for 60 minutes;

f. in “quenching” plate TO, 180 μL of quenching solution and 6 μL of NAPDH cofactor were addded to make sure that the plate was cooled down to prevent evaporation;

g. for T60 plates, the mixture was fully mixed for three times, and 54 μL of mixture was immediately transferred to the “quenching” plate at the 0 min time point. 44 μL of NAPDH cofactor was then added to the culture plate (T60). Timing was started; the plate was shaken at 37° C. for 60 minutes;

h. at 5, 10, 20, 30 and 60 min, 180 μL of quenching solution was added to the “quenching” plate, and the mixture was mixed for one time, and 60 μL of sample was continuously transferred from the T60 plate to the “quenching” plate at each time point;

i. for NCF60: the mixture was mixed for one time, and 60 μL of sample was transferred from the NCF60 culture dish to the “quenching” plate containing quenching solution at 60 minutes time point;

j. all sampling plates were shaked for 10 minutes, then centrifuged at 4000 rpm for 20 minutes at 4° C.;

k. 60 μL of supernatant was transferred into 180 μL of HPLC water and stirred with a plate shaker for 10 min;

1. before LC-MS/MS analysis, each bioanalysis plate was sealed and shaked for 10 minutes.

The experimental results are shown in table 3 and table 4:

TABLE 3
In vitro human and SD rat liver microsome
metabolic stability data for compound 2

| | Human | | | SD rat | | |
|---|---|---|---|---|---|---|
| | T_{1/2} | C_{Lint}(liver) | Remaining | T_{1/2} | C_{Lint}(liver) | Remaining |
| Name | (min) | (mL/min/kg) | (T = 60 min) | (min) | (mL/min/kg) | (T = 60 min) |
| Compoundn 2 | 52.2 | 23.9 | 45.6% | 105.6 | 23.6 | 64.1% |
| Testosterone | 8.9 | 155 | 5.7% | 1.1 | 2182.3 | 0.0% |
| Diclofenac | 4.5 | 309 | 0.0% | 14.4 | 173.8 | 5.2% |
| Propafenone | 5.9 | 235.1 | 0.1% | 1.0 | 2590.4 | 0.0% |

TABLE 4
In vitro CD-1 mouse and beagle dog liver microsome
metabolic stability data for compound 2

| | CD-1 mouse | | | Beagle dog | | |
|---|---|---|---|---|---|---|
| | T_{1/2} | C_{Lint}(liver) | Remaining | T_{1/2} | C_{Lint}(liver) | Remaining |
| Name | (min) | (mL/min/kg) | (T = 60 min) | (min) | (mL/min/kg) | (T = 60 min) |
| Compound 2 | 68.8 | 79.7 | 54.6% | 74.4 | 26.8 | 52% |
| Testosterone | 4.0 | 1368.5 | 0.0% | 24.6 | 81.0 | 19% |
| Diclofenac | 63.5 | 86.4 | 53.0% | >145 | <13.8 | 101% |
| Propafenone | 2.0 | 2720.1 | 0.1% | 5.4 | 369.3 | 0% |

Note:
T_{1/2} indicates half-life; C_{Lint} (liver) indicates the intrinsic clearance rate of liver microsomes; Remaining (T = 60 min) indicates the remaining rate of the compound after 60 minutes of incubation.

Conclusion: The results show that the compounds of the present disclosure have good stability in the liver microsomal metabolism of humans, SD rats, CD-1 mice and beagle dogs, and the species differences are small.

### Experimental Embodiment 4: In Vivo Pharmacokinetic Property Evaluation in Mice

The in vivo pharmacokinetic properties of compound 2 were evaluated in CD-1 mice by intravenous administration and oral administration. IV (intravenous injection) refers to slow administration in jugular vein, and PO (oral administration) refers to administration by gavage. Formulations for intravenous and gavage administration were both 2.5% dimethylsulfoxide, 5% ethanol, 10% cremophor EL, 20% glucose solution (concentration of 5%), 62.5% water. PK time points in the intravenous injection group were 5 min, 15 min, 30 min, 1 h, 2 h, 4 h, 8 h, 12 h and 24 h after administration, respectively, and PK time points in the gastric administration group were 15 min, 30 min, 1 h, 2 h, 4 h, 8 h and 24 h after administration, respectively. Approximately 0.03 mL of blood was collected at each time point. Blood from each sample was transferred to a plastic microcentrifuge tube containing EDTA-K2, centrifuged at 4000 rpm for 5 min in a 4° C. centrifuge, and plasma was collected within 15 min, and the plasma samples were stored in polypropylene tubes. Before the test, samples were stored in a refrigerator at −75±15° C. The concentration of compounds in plasma samples was analyzed using the LCMS/MS method and the following pharmacokinetic parameters were calculated using WinNonlin software (Phoenix™, version 6.1): IV: C₀, Cl, V_{d}, T_{1/2}, AUC₀₋ₗₐₛₜ, MRT₀₋ₗₐₛₜ, regression points; PO: Cₘₐₓ, Tₘₐₓ, T_{1/2}, AUC₀₋ₗₐₛₜ, MRT₀₋ₗₐₛₜ, regression points. Pharmacokinetic data were described using descriptive statistics, such as mean, standard deviation.

The statistical results are shown in table 5 and table 6:

TABLE 5
Pharmacokinetic data of compound 2 (IV, 1 mpk) administered to CD-1 mice

| | C₀ | T_{1/2} | Vdₛₛ | Cl | AUC₀₋ₗₐₛₜ | MRT₀₋ₗₐₛₜ |
|---|---|---|---|---|---|---|
| Parameter | (nmol/L) | (h) | (L/kg) | (mL/min/kg) | (h*nmol/L) | (h) |
| Test value | 11669 | 3.35 | 0.834 | 10.2 | 1799 | 1.21 |

TABLE 6
Pharmacokinetic data of compound 2 (PO,
50 mpk) administered to CD-1 mice

| | Cₘₐₓ | Tₘₐₓ | T_{1/2} | AUC₀₋ₗₐₛₜ | MRT₀₋ₗₐₛₜ |
|---|---|---|---|---|---|
| Parameter | (nmol/L) | (h) | (h) | (h*nmol/L) | (h) |
| Test value | 1790 | 3.0 | 4.1 | 12809 | 6.22 |

Note:
C₀ indicates the drug concentration at the initial time point; T_{1/2} indicates half-life; Vdₛₛ indicates the apparent distribution volume; Cl indicates plasma clearance rate; AUC₀₋ₗₐₛₜ indicates the plasma exposure of the drug; MRT₀₋ₗₐₛₜ indicates the average residence time; Cₘₐₓ indicates the maximum drug concentration point; Tₘₐₓ indicates the peak time.

Conclusion: The compounds of the present disclosure have good pharmacokinetic properties in CD-1 mice in vivo, supporting the oral administration route.

### Experimental Embodiment 5: In Vivo Anti-Tumor Effect on BALB/c Nude Mouse Human Acute Lymphoblastic Leukemia Cell RS4;11 Graft Tumor Model

5.1 Experimental animals: Balb/c nude mice, 32 mice, 7 to 8 weeks old, female;

5.2 tumor cells: human acute lymphoblastic leukemia cell line RS4;11, cultured in a suspension in vitro, and the culture conditions were RPMI-1640 culture medium containing 10% fetal bovine serum, cultured in a 5% CO₂ incubator at 37° C. When the cells were in exponential growth period and the saturation was 80% to 90%, the cells were collected and counted;

5.3 cell seeding and grouping: the cells were resuspended in sodium dihydrogen phosphate buffer solution, and the basement membrane matrigel was added in 1:1, and the mixture was mixed well, and the density was 5×10⁷ cells/mL. 0.2 mL of cell suspension (containing 1×10⁷ RS4;11 cells) was subcutaneously inoculated on the right back of each mouse, and when the average tumor volume reached about 120 mm³, the drug was administered randomly according to the tumor volume;

5.4 preparation of the subject: an appropriate amount of compound 2 was weighed respectively, and the solvent formula was 2.5% dimethyl sulfoxide, 5% ethanol, 10% cremophor EL, 20% glucose solution (concentration of 5%), 62.5% water;

5.5 tumor-bearing mice divided into four groups (8 mice in each group) were given blank solvent, compound 2 (12.5 mpk, QD), compound 2 (25 mpk, QD) and compound 2 (50 mpk, QD), respectively;

5.6 tumor measurement and experimental indicators:

tumor diameters were measured with vernier calipers two times a week. The calculation formula of tumor volume is: V=0.5×a×b², a represents the long diameter of the tumor, and b represents the short diameter of the tumor. The tumor inhibition efficacy of the compounds was evaluated by TGI (%) or relative tumor proliferation rate T/C (%).

Relative tumor proliferation rate T/C %=T_{RTV}/C_{RTV}×100% (T_{RTV}: RTV in the treatment group; C_{RTV}: RTV in the negative control group). The relative tumor volume (RTV) is calculated according to the results of tumor measurement, and the formula is RTV=Vₜ/V₀, where V₀ is the tumor volume measured at the time of group administration (i.e., D₀), and Vₜ is the tumor volume measured at a certain measurement, T_{RTV} and C_{RTV} were taken on the same day.

TGI (%), reflecting the tumor growth inhibition rate. The formula for calculating the tumor inhibition efficacy TGI is:

$$Tumor inhibition efficacy TGI ( \% ) = ( 1 - \frac{\begin{matrix}average tumor volume at the end of \\ administration in a treatment group - \\ average tumor volume at the beginning of \\ administration in this treatment group\end{matrix}}{\begin{matrix}average tumor volume at the end of \\ treatment in a solvent control group - \\ average tumor volume at the beginning of \\ treatment in this solvent control group\end{matrix}} ) \times 100 \%$$

The statistical results are shown in table 7 and table 8:

TABLE 7
Effects of subjects on tumor volume (mm³) administered for different days

| | Tumor volume (mm³) | | | |
|---|---|---|---|---|
| Days after | Solvent control | Compound 2 | Compound 2 | Compound 2 |
| administration | group | (12.5 mpk, QD) | (25 mpk, QD) | (50 mpk, QD) |
| 1 | 114.80 ± 5.34 | 114.44 ± 4.91 | 114.50 ± 4.98 | 114.18 ± 4.30 |
| 4 | 138.76 ± 8.89 | 124.75 ± 10.85 | 99.54 ± 6.66 | 95.13 ± 7.43 |
| 6 | 196.14 ± 12.92 | 120.60 ± 12.60 | 106.03 ± 13.15 | 91.16 ± 6.08 |
| 8 | 203.61 ± 16.61 | 124.22 ± 15.25 | 107.66 ± 15.62 | 86.91 ± 4.92 |
| 11 | 218.27 ± 17.86 | 126.73 ± 11.57 | 101.57 ± 15.87 | 67.64 ± 3.93 |
| 14 | 278.56 ± 29.49 | 145.41 ± 17.58 | 116.52 ± 28.85 | 74.27 ± 6.37 |
| 18 | 449.58 ± 28.79 | 198.17 ± 38.18 | 146.77 ± 44.03 | 58.39 ± 6.94 |
| 21 | 577.26 ± 49.88 | 209.63 ± 39.56 | 191.06 ± 69.68 | 70.21 ± 8.91 |
| 25 | 862.06 ± 84.07 | 254.52 ± 52.29 | 174.50 ± 58.56 | 75.62 ± 10.30 |
| 28 | 1117.50 ± 93.76 | 306.93 ± 66.01 | 206.82 ± 76.64 | 78.03 ± 9.52 |
| 32 | 1516.64 ± 180.61 | 365.13 ± 85.31 | 266.63 ± 95.51 | 110.37 ± 19.73 |

TABLE 8
Tumor inhibition efficacy of test compounds on
RS4; 11 xenograft model (based on tumor volume
data on the day 32 after administration)

| | Tumor volume* (mm³) | | T/C | TGI |
|---|---|---|---|---|
| Group | Day 1 | Day 32 | (%) | % |
| Oral administration of | 114.80 ± | 1516.64 ± | — | — |
| solvent control group | 5.34 | 180.61 | | |
| Compound 2 (12.5 mpk, QD) | 114.44 ± | 365.13 ± | 22.37 | 82.12 |
| | 4.91 | 85.31 | | |
| Compound 2 (25 mpk, QD) | 114.50 ± | 266.63 ± | 16.54 | 89.15 |
| | 4.98 | 95.51 | | |
| Compound 2 (50 mpk, QD) | 114.18 ± | 110.37 ± | 7.30 | 100.27 |
| | 4.30 | 19.73 | | |

Note
*Mean ± SEM, n = 8.

Conclusion: According to the data of tumor volume and tumor inhibition efficacy, the three dose groups of compound 2 all show significant tumor inhibition effect, and show obvious dose-dependence. The higher the dose, the more significant the tumor inhibition effect, and the animal state is normal during the experiment.

1. A compound represented by formula (I) or a pharmaceutically acceptable salt thereof, 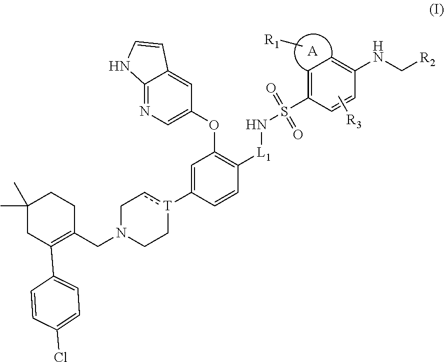
wherein,
when T is N, [symbol] is selected from a single bond;
when T is C, [symbol] is selected from a double bond;
ring A is selected from 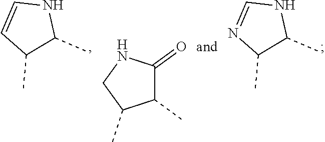
R₁ is selected from H and C₁₋₃ alkyl, and the C₁₋₃ alkyl is optionally substituted by one Rₐ;
R₂ is selected from oxacyclohexyl;
R₃ is selected from H, F, Cl, Br, I, NO₂ and CN;
L₁ is selected from a single bond and —C(═O)—;
Rₐ is selected from H and

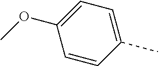

2. The compound or the pharmaceutically acceptable salt thereof as claimed in claim 1, wherein, the R₁ is selected from H and CH₃, and the CH₃ is optionally substituted by one Rₐ.

3. The compound or the pharmaceutically acceptable salt thereof as claimed in claim 2, wherein, the R₁ is selected from H, CH₃ and

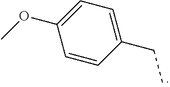

4. The compound or the pharmaceutically acceptable salt thereof as claimed in claim 1, wherein, the R₂ is selected from

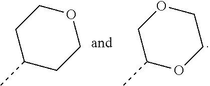

5. The compound or the pharmaceutically acceptable salt thereof as claimed in claim 1, wherein, the R₃ is selected from H and NO₂.

6. The compound or the pharmaceutically acceptable salt thereof as claimed in claim 1, wherein, the compound is selected from 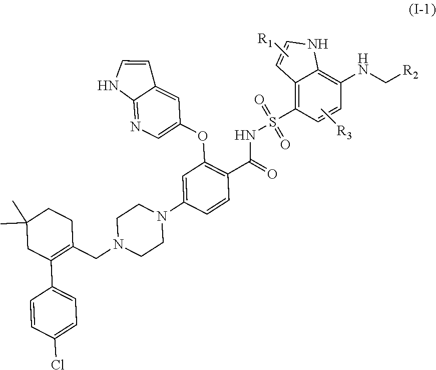
wherein, R₁, R₂ and R₃ are as defined above.

7. The compound or the pharmaceutically acceptable salt thereof as claimed in claim 1, wherein, the compound is selected from 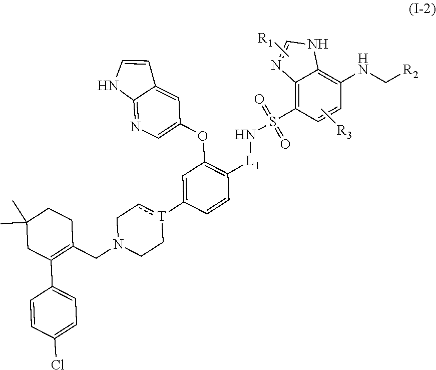
wherein,
when T is N, [symbol] is selected from a single bond;
when T is C, [symbol] is selected from a double bond;
R₁, R₂, R₃ and L₁ are as defined above.

8. The compound or the pharmaceutically acceptable salt thereof as claimed in claim 1, wherein, the compound is selected from 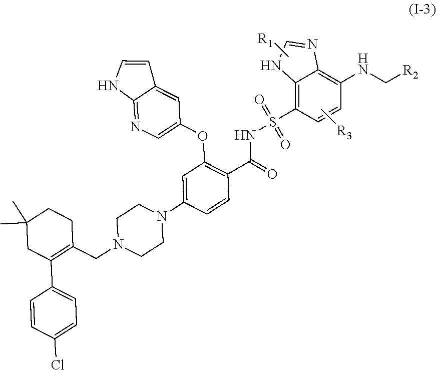
wherein, R₁, R₂ and R₃ are as defined above.

9. The compound or the pharmaceutically acceptable salt thereof as claimed in claim 1, wherein, the compound is selected from 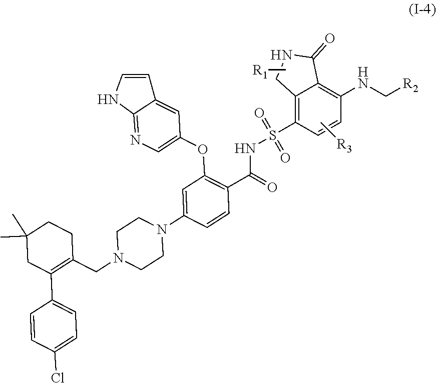
wherein, R₁, R₂ and R₃ are as defined above.

10. The compound or the pharmaceutically acceptable salt thereof as claimed in claim 1, wherein, the structural moiety 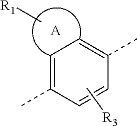
is selected from 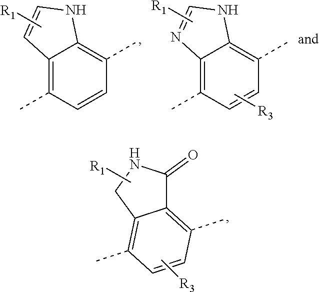
wherein, R₁ and R₃ are as defined above.

11. The compound or the pharmaceutically acceptable salt thereof as claimed in claim 10, wherein, the structural moiety 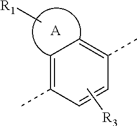
is selected from 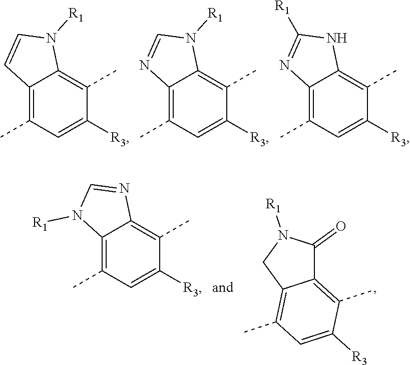
wherein, R₁ and R₃ are as defined above.

12. The compound or the pharmaceutically acceptable salt thereof as claimed in claim 11, wherein, the structural moiety 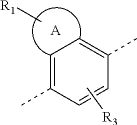
is selected from

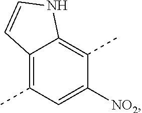

13. A compound represented by the following formula or a pharmaceutically acceptable salt thereof is selected from,

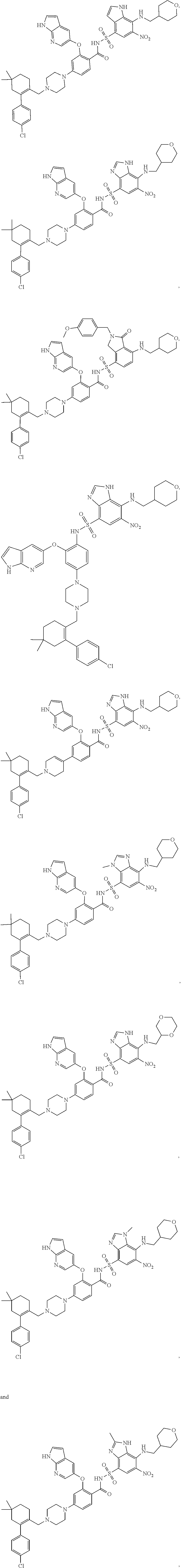

14. A method of inhibiting Bcl-2 in a subject in need thereof, comprising administering a therapeutically effective amount of the compound or the pharmaceutically acceptable salt thereof as defined in claim 1 into the subject.

15. A method of treating hematological malignancies and solid tumors in a subject in need thereof, comprising administering a therapeutically effective amount of the compound or the pharmaceutically acceptable salt thereof as defined in claim 1 into the subject.
